SEQUENCE LISTING (41 sequences)

SEQ ID NO: 1 - DNA, 1761 bp, Homo sapiens
ctcgagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggccttcgtggcctcgag

SEQ ID NO: 2 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 3 - DNA, 1787 bp, Homo sapiens
ctcgagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggccttcgtggcctcgag

SEQ ID NO: 4 - protein, 261 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 5 - DNA, 26 bp, Homo sapiens
ctcagactacgtgcacctctgcagca

SEQ ID NO: 6 - protein, 210 aa, Homo sapiens
GAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 7 - protein, 95 aa, Homo sapiens
LRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 8 - DNA, 24 bp, Artificial Sequence
  Primer
aaacagagtagcagctcagactgc

SEQ ID NO: 9 - DNA, 22 bp, Artificial Sequence
  Primer
tgggcagtggctggatgaaagc

SEQ ID NO: 10 - DNA, 24 bp, Artificial Sequence
  Primer
aaacagagtagcagcgcagactgc

SEQ ID NO: 11 - DNA, 22 bp, Artificial Sequence
  Primer
cagacaatggctggatgaaagc

SEQ ID NO: 12 - DNA, 24 bp, Artificial Sequence
  Primer
aaacagagtagcagcacagactgc

SEQ ID NO: 13 - DNA, 20 bp, Artificial Sequence
  Primer
cagcactcagactacgtgca

SEQ ID NO: 14 - DNA, 21 bp, Artificial Sequence
  Primer
atccatggggagatgttctgg

SEQ ID NO: 15 - DNA, 21 bp, Artificial Sequence
  Primer
ctgagtccgaatcaggtgcag

SEQ ID NO: 16 - DNA, 20 bp, Artificial Sequence
  Primer
cagcactcagactatgtgca

SEQ ID NO: 17 - DNA, 21 bp, Artificial Sequence
  Primer
gtccatgggaagatgttctgg

SEQ ID NO: 18 - DNA, 21 bp, Artificial Sequence
  Primer
atccatgggaagatgttctgg

SEQ ID NO: 19 - DNA, 20 bp, Artificial Sequence
  Primer
cagcactcagactacgtgcg

SEQ ID NO: 20 - protein, 12 aa, Artificial Sequence
  Synthetically generated peptide
CVGRHXFSRRSG

SEQ ID NO: 21 - protein, 12 aa, Artificial Sequence
  Synthetically generated peptide
CVGRHSFSRRSG

SEQ ID NO: 22 - DNA, 19 bp, Artificial Sequence
  Primer
caagtgtgggtacgccacg

SEQ ID NO: 23 - DNA, 21 bp, Artificial Sequence
  Primer
aaagaagctctccatccggtc

SEQ ID NO: 24 - DNA, 18 bp, Artificial Sequence
  Primer
ctacctgcgaagaggccg

SEQ ID NO: 25 - DNA, 20 bp, Artificial Sequence
  Primer
gttcatgagctgcccactga

SEQ ID NO: 26 - DNA, 20 bp, Artificial Sequence
  Primer
ccatcaacatgctcccgttc

SEQ ID NO: 27 - DNA, 19 bp, Artificial Sequence
  Primer
gggtaggcctcgccataca

SEQ ID NO: 28 - protein, 15 aa, Artificial Sequence
  Synthetically generated peptide
TCRMDVLRASLPAQS

SEQ ID NO: 29 - DNA, 24 bp, Artificial Sequence
  Primer
ttcagccaattatcagcaaactct

SEQ ID NO: 30 - DNA, 26 bp, Artificial Sequence
  Primer
ttttctgatgtatcctcttcaccagt

SEQ ID NO: 31 - DNA, 21 bp, Artificial Sequence
  Primer
cctgcttgtgagtatgggacc

SEQ ID NO: 32 - DNA, 20 bp, Artificial Sequence
  Primer
tgggtttccacagttgggaa

SEQ ID NO: 33 - DNA, 23 bp, Artificial Sequence
  Primer
acagccttaaccaacttgaggtg

SEQ ID NO: 34 - DNA, 20 bp, Artificial Sequence
  Primer
tccgggaagcaacgaatcta

SEQ ID NO: 35 - DNA, 21 bp, Artificial Sequence
  Synthetically generated oligonucleotide
aaggccatgatctccgacttt

SEQ ID NO: 36 - DNA, 21 bp, Artificial Sequence
  Synthetically generated oligonucleotide
aaggagctttgaggaagttat

SEQ ID NO: 37 - DNA, 21 bp, Artificial Sequence
  Synthetically generated oligonucleotide
aaggcgatgatctcagacttt

SEQ ID NO: 38 - DNA, 3071 bp, Homo sapiens
gggggtggggagtgttgttaaccggaggggcagccgcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcctctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcttttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaaaaaaaaaa

SEQ ID NO: 39 - protein, 616 aa, Homo sapiens
MFRTAVMMAASLALTGAVVAHAYYLKHQFYPTVVYLTKSSPSMAVLYIQAFVLVFLLGKVMGKVFFGQLRAAEMEHLLERSWYAVTETCLAFTVFRDDFSPRFVALFTLLLFLKCFHWLAEDRVDFMERSPNISWLFHCRIVSLMFLLGILDFLFVSHAYHSILTRGASVQLVFGFEYAILMTMVLTIFIKYVLHSVDLQSENPWDNKAVYMLYTELFTGFIKVLLYMAFMTIMIKVHTFPLFAIRPMYLAMRQFKKAVTDAIMSRRAIRNMNTLYPDATPEELQAMDNVCIICREEMVTGAKRLPCNHIFHTSCLRSWFQRQQTCPTCRMDVLRASLPAQSPPPPEPADQGPPPAPHPPPLLPQPPNFPQGLLPPFPPGMFPLWPPMGPFPPVPPPPSSGEAVAPPSTSAALSRPSGAATTTAAGTSATAASATASGPGSGSAPEAGPAPGFPFPPPWMGMPLPPPFAFPPMPVPPAGFAGLTPEELRALEGHERQHLEARLQSLRNIHTLLDAAMLQINQYLTVLASLGPPRPATSVNSTEETATTVVAAASSTSIPSSEATTPTPGASPPAPEMERPPAPESVGTEEMPEDGEPDAAELRRRRLQKLESPVAH

SEQ ID NO: 40 - DNA, 3074 bp, Homo sapiens
gggggtggggagtgttgttaaccggaggggcagccgcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcctctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcttttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaaaaaaaaaa

SEQ ID NO: 41 - protein, 617 aa, Homo sapiens
MFRTAVMMAASLALTGAVVAHAYYLKHQFYPTVVYLTKSSPSMAVLYIQAFVLVFLLGKVMGKVFFGQLRAAEMEHLLERSWYAVTETCLAFTVFRDDFSPRFVALFTLLLFLKCFHWLAEDRVDFMERSPNISWLFHCRIVSLMFLLGILDFLFVSHAYHSILTRGASVQLVFGFEYAILMTMVLTIFIKYVLHSVDLQSENPWDNKAVYMLYTELFTGFIKVLLYMAFMTIMIKVHTFPLFAIRPMYLAMRQFKKAVTDAIMSRRAIRNMNTLYPDATPEELQAMDNVCIICREEMVTGAKRLPCNHIFHTSCLRSWFQRQQTCPTCRMDVLRASLPAQSPPPPEPADQGPPPAPHPPPLLPQPPNFPQGLLPPFPPGMFPLWPPMGPFPPVPPPPSSGEAVAPPSTSAAALSRPSGAATTTAAGTSATAASATASGPGSGSAPEAGPAPGFPFPPPWMGMPLPPPFAFPPMPVPPAGFAGLTPEELRALEGHERQHLEARLQSLRNIHTLLDAAMLQINQYLTVLASLGPPRPATSVNSTEETATTVVAAASSTSIPSSEATTPTPGASPPAPEMERPPAPESVGTEEMPEDGEPDAAELRRRRLQKLESPVAH

(19) United States
(12) Patent Application Publication
(10) Pub. No.: US 2010/0221743 A1
(43) Pub. Date: Sep. 2, 2010

(54) Methods for Diagnosing and Treating Endoplasmic Reticulum (ER) Stress Diseases

(75) Inventor: Fumihiko Urano, Wellesley, MA (US)

Correspondence Address: FISH & RICHARDSON PC, P.O. BOX 1022, MINNEAPOLIS, MN, 55440-1022, US

(73) Assignee: UNIVERSITY OF MASSACHUSETTS, Shrewsbury, MA (US)

(21) Appl. No.: 12/718,719

(22) Filed: Mar. 5, 2010

Related U.S. Application Data
(62) Division of application No. 10/574,194, filed on Mar. 2, 2007, now abandoned.
(60) Provisional application No. 60/510,262, filed on Oct. 9, 2003.
(60) Provisional application No. 60/519,736, filed on Nov. 12, 2003.
(60) Provisional application No. 60/568,468, filed on May 5, 2004.

(51) Int. Cl. G01N 33/53; C07K 16/00

(52) U.S. Cl. 435/7.1; 530/389.1

(57) ABSTRACT

The present invention provides methods and reagents to quantify endoplasmic reticulum stress (ER stress) levels, and methods and compounds for treating ER stress disorders such as diabetes. Methods for quantifying ER stress in mammalian cells are exemplified.

### CLAIM OF PRIORITY

This application is a divisional of U.S. patent application Ser. No. 10/574,194, which is the National Phase of International Patent Application No. PCT/US2004/033516, filed on Oct. 12, 2004, and claims the benefit of U.S. Provisional Patent Application Ser. Nos. 60/510,262, filed on Oct. 9, 2003; 60/519,736, filed on Nov. 12, 2003; and 60/568,468, filed on May 5, 2004. The entire contents of the foregoing are hereby incorporated by reference herein.

### FEDERALLY SPONSORED RESEARCH OR DEVELOPMENT

This invention was made, at least in part, with government support under grants no. R01 DK067493-01 and DK32520, awarded by the National Institute of Diabetes and Digestive and Kidney Diseases of the National Institutes of Health. The government has certain rights in this invention.

### BACKGROUND

Proteins are required for the body to function properly, as they form the basic building blocks of cells, tissues and organ structures. Protein function typically requires the assumption of proper three-dimensional protein structure, which is determined by the amino acid sequence of the protein and a process known as protein folding. Sometimes, protein folding goes awry, and misfolded proteins accumulate in cells, causing or contributing to diseases associated with protein misfolding, including amyloidoses (such as immunoglobulin light chain amyloidosis and Alzheimer's disease), Huntington's disease, Parkinson's disease, adult-onset diabetes mellitus, cirrhosis, emphysema, prion encephalopathies, alpha-1-antitrypsin deficiency, hemolytic anemia, familial hypercholesterolaemia, amyotrophic lateral sclerosis (ALS), and cystic fibrosis, as well as numerous others. Conformational diseases can be inherited, usually as dominant traits, or can be induced, as in the case of prions.

Proteins destined for secretion such as insulin and alpha1-antitrypsin are translocated into the endoplasmic reticulum (ER) co-translationally; once there, they undergo highly ordered protein folding and post-translational protein processing. However, in some instances, the sensitive folding environment in the ER can be perturbed by pathophysiological processes such as viral infections, environmental toxins, and mutant protein expression, as well as natural processes such as the large biosynthetic load placed on the ER. When the demand that the load of proteins makes on the ER exceeds the actual folding capacity of the ER to meet that demand, a condition termed “ER stress” results.

Alpha1-antitrypsin (alpha1-AT) deficiency is an exemplary model of a conformational disease. Alpha1-AT is an abundant serum glycoprotein, secreted by the liver, which normally binds to and inactivates elastase, a protease that degrades elastin and collagen. Elastin and collagen maintain the structure of alveoli, air sacs in the lungs. In alpha1-antitrypsin patients, the deficiency leads to uncontrolled destruction of air sacs in the lungs. This condition is called emphysema and causes a decrease in respiratory function. Alpha1-AT-deficiency mutations interfere with the folding of alpha1-AT, preventing its secretion from the hepatocyte ER. Alpha1-AT deficiency is also the leading cause of inherited liver disease in children, caused by the hepatotoxicity of misfolded alpha1-AT molecules that accumulate in the ER lumen.

Cells respond to the accumulation of misfolded proteins in the ER in several ways, including the “ER overload response” and the “unfolded protein response.” The “ER overload response” induces the nuclear transcription factor NF-κB, a mediator of the immune response. In patients with cystic fibrosis, expression of mutant CFTR induces NF-κB expression. NF-kappaB upregulates expression of the inflammatory cytokine IL8. Levels of IL-8 are increased in lungs of patients with cystic fibrosis, and NF-κB was found to be constitutively active in mice in which the wild-type CFTR gene had been replaced with the F508 mutant, supporting the theory that ER stress contributes to the chronic inflammation that often contributes to the high morbidity in cystic fibrosis.

The “unfolded protein response” (UPR), triggered by the presence of misfolded protein in the ER, consists of three components that counteract ER stress: gene expression, translational attenuation, and ER-associated protein degradation (the ERAD system) (Harding et al., Ann. Rev. Cell Dev. Biol. 18:575-599 (2002); Kaufman et al., Nat. Rev. Mol. Cell. Biol. 3:411-421 (2002); Mori, Cell, 101:451-454 (2000)). In particular, the ERAD system has an important function in the survival of stressed cells (Yoshida et al., Dev. Cell 4:265-271 (2003); Kaneko et al., FEBS Lett. 532:147-152 (2002)). It has been shown that inositol requiring 1 (IRE1), a crucial regulator of the ERAD system (Yoshida et al., 2002, supra), is a sensor for unfolded and misfolded proteins in the ER. The presence of unfolded or misfolded proteins in the ER causes dimerization and trans-autophosphorylation of IRE1, leading to IRE1 activity. Activated IRE1 splices the X-box-binding protein-1 (XBP-1) mRNA, leading to synthesis of the active transcription factor XBP-1 and upregulation of UPR genes, particularly ERAD genes (Yoshida et al., 2002, supra; Calfon et al., Nature 415:92-96 (2002)).

### SUMMARY

The present invention provides novel methods and reagents for quantifying levels of endoplasmic reticulum (ER) stress, and for diagnosing and treating ER stress disorders. In some embodiments, the methods feature the use of Inositol Requiring 1 (IRE1) and/or X-box-binding protein-1 (XBP-1) as specific markers for ER stress level. It can be difficult to directly measure the activity level of IRE1, because although activation of IRE1 by phosphorylation causes a shift to lower mobility on an SDS-polyacrylamide gel, the shift is very small and thus difficult to detect. Because of this difficulty, XBP-1 mRNA splicing levels, which precisely reflect IRE1 activity, can be used to quantify ER stress levels. Exemplary methods are based on PCR. For these methods, only a small tissue sample or a small number of cells are required. Alternatively, an antibody specific for the phosphorylated form or IRE1, such as is described herein, can be used to detect IRE1 activity levels. These methods can be used to diagnose ER stress disorders and to identify novel therapeutic modalities, e.g., new therapeutic agents, for the treatment of ER stress disorders.

Thus, in one aspect, the invention provides methods of quantifying ER stress. The methods include detecting an IRE1 activity level in a cell or biological sample, wherein the IRE1 activity level correlates with ER stress, and quantifying the IRE1 activity level, such that ER stress is quantified. An increase in IRE1 activity indicates an increase in ER stress, and a decrease in IRE1 activity indicates a decrease in ER stress. In some embodiments, the methods include comparing the level of ER stress, e.g., the level of IRE1 activity, with a reference, and an increase in the level of ER stress as compared to the reference indicates the presence of ER stress, e.g., an ER stress disease.

In some embodiments, the IRE1 activity level is determined by detecting an XBP-1 splicing level, e.g., by amplifying a XBP-1 mRNA region that includes a splice site, or portion thereof, e.g., to create a DNA complementary to the region of the XBP-1 mRNA, e.g., a double-stranded cDNA PCR product; detecting the size of the amplified mRNA (e.g., the cDNA), wherein the size is indicative of spliced or unspliced mRNA. In some embodiments, the level of spliced XBP-1 are detected and/or the level of unspliced XBP-1 are detected. In some embodiments, both the level of spliced XBP-1 and the level of unspliced XBP-1 are detected, and the ratio of spliced XBP-1 to unspliced XBP-1 is determined. In some embodiments, the amplified mRNA is subjected to restriction enzyme digestion, e.g., Pst I digestion, to facilitate detection of spliced or unspliced mRNA.

In some embodiments, the IRE1 activity level is determined by detecting levels of IRE1 autophosphorylation. In some embodiments, the IRE1 activity level is determined by detecting the percentage or ratio of autophosphorylated to unphosphorylated IRE1.

In some embodiments, the ER stress level is quantified in a cell, e.g., a mammalian cell, e.g., a human cell, e.g., a pancreatic beta cell. In some embodiments, the ER stress level is quantified in a cell extract, e.g., an extract from a cell as described herein.

In another aspect, the invention provides methods of diagnosing an ER stress disorder, e.g., diabetes or Wolfram Syndrome, in a subject by quantifying the level of ER stress in a cell or biological sample isolated from the subject according to one of the methods described herein. An increased level of ER stress, e.g., as compared to a suitable control, is indicative of the ER stress disorder. In some embodiments, the cell or biological sample comprises a peripheral blood cell, e.g., a lymphocyte.

The invention also provides methods of monitoring the progression of an ER stress disorder, e.g., diabetes, in a subject. The methods include quantifying the level of ER stress in a cell or biological sample isolated from the subject at sequential time points according to one of the methods described herein, wherein a change in the level of ER stress indicates the progress of the ER stress disorder. An increased level of ER stress, e.g., as compared to a suitable control, e.g., the level of ER stress in a sample from the same subject at an earlier time point, indicates that the disorder is progressing. A decreased level of ER stress can indicate that the disorder is in remission, or that a treatment is effective.

Further, the invention includes methods for identifying modulators of ER stress. The methods include providing a providing an ER stress model system (e.g., a system comprising a cell expressing WFS1 (the Wolfram Syndrome 1 gene, sometimes referred to as Wolframin; OMIM No. 606201), IRE1 (Inositol-Requiring 1, sometimes referred to as endoplasmic reticulum-to-nucleus signaling 1, ERN1; OMIM No. 604033) and/or XBP-1 (X box-binding protein 1; OMIM No. 194355), e.g., a cultured cell or animal, e.g., a cell or animal model of an ER stress disorder); optionally, increasing levels of ER stress in the system (e.g., in the cells or at least some of the cells of an animal); contacting the system with a test compound; and evaluating the levels of ER stress in the system in the presence and absence of the test compound. In some embodiments levels of ER stress are evaluated by measuring XBP-1 splicing, wherein an increase in XBP-1 splicing indicates an increase in ER stress, and a decrease in XBP-1 splicing indicates a decrease in ER stress. In other embodiments, levels of ER stress are evaluated by detecting levels of IRE1 autophosphorylation, wherein an increase in IRE1 autophosphorylation indicates an increase in ER stress, and a decrease in IRE1 autophosphorylation indicates a decrease in ER stress. An “increase” or “decrease” can be determined relative to a suitable control.

In a further aspect, the invention provides methods for identifying candidate compounds that reduce ER stress. The methods include providing an ER stress model system; optionally, increasing ER stress in the system; contacting the system with a test compound; and evaluating a level of HRD1 activity in the system in the presence and absence of the test compound. An increase in the level of HRD1 activity indicates that the test compound is a candidate compound that reduces ER stress. In some embodiments, the method also includes contacting an ER stress model system with a candidate compound that increases HRD1 activity; and evaluating ER stress in the system in the presence of the candidate compound, wherein a decrease in ER stress in the system in the presence of the candidate compound indicates that the candidate compound is a candidate therapeutic agent for the treatment of an ER stress disorder.

In some embodiments, the model is an animal model; in some embodiments, the method includes contacting the model with a candidate therapeutic agent for the treatment of an ER stress disorder identified by a method described herein; and evaluating the levels of ER stress in the system in the presence of the candidate compound. An improvement in the model in the presence of the candidate therapeutic agent indicates that the agent is a therapeutic agent for the treatment of an ER stress disorder.

In some embodiments, the compound or agent is a nucleic acid, polypeptide, peptide, or small molecule, e.g., an HRD1 nucleic acid, polypeptide, or a functional fragment thereof, e.g., the functional fragment is or encodes a peptide comprising the cytosolic RING-H2 domain of HRD1 or a homolog thereof, a peptide comprising amino acids 291-333 of SEQ ID NOs:39 or 41, or a peptide comprising amino acids 272-243 of SEQ ID NOs: 39 or 41.

In some embodiments, the system is an animal model of an ER stress disorder, e.g., an animal model of diabetes (e.g., type 1 or type 2 diabetes), Alzheimer's disease, Parkinson's disease, Wolfram Syndrome, Cystic Fibrosis, familial hypercholesterolaemia, or alpha1 antitrypsin deficiency, or cells derived therefrom. Typically, an ER stress disorder can be induced in an otherwise healthy animal or cells by administering a compound known to cause ER dysfunction, e.g., by administering a sublethal dose of thapsigargin, tunicamycin (e.g., 0.25-1 mg/kg tunicamycin), or a proteosome inhibitor, e.g., lactacystin.

In some embodiments, the methods include further selecting those test compounds that substantially reduce ER stress (e.g., as measured by IRE1 autophosphorylation levels or XBP-1 splicing levels) as candidate therapeutic compounds for further evaluation.

Also described herein is a kit for quantifying ER stress. The kit can include primers for amplifying a region of XBP-1 mRNA that includes a splice site, or portion thereof, and instructions for use. In some embodiments, the kit also includes a suitable control. In one embodiment, the kit includes one or more primers for amplifying a region of XBP-1 mRNA that includes a splice site, or portion thereof; one or more of: a control comprising a spliced XBP-1 nucleic acid and a control comprising an unspliced XBP-1 nucleic acid; and instructions for use.

The invention further includes antibodies that bind specifically to the autophosphorylated form of IRE1, and do not substantially bind the unphosphorylated form. The antibodies can be polyclonal, monoclonal, or monospecific, or antigen-binding fragments thereof.

The invention also includes an ER stress signaling pathway assay that includes determining the level of ER stress according to one of the methods described herein.

Further, the invention includes therapeutic compositions for the treatment of an ER stress disorder. In some embodiments, the therapeutic composition includes an HRD1 nucleic acid, polypeptide, or a functional fragment thereof and a pharmaceutically acceptable carrier, e.g., the functional fragment is or encodes a peptide comprising the cytosolic RING-H2 domain of HRD1 or a homolog thereof, a peptide comprising amino acids 291-333 of SEQ ID NOs: 39 or 41, or a peptide comprising amino acids 272-243 of SEQ ID NOs: 39 or 41.

The invention also provides methods of treating subjects having or at risk of an ER stress disorder, by administering to the subject a therapeutically effective amount of a therapeutic agent identified by a method described herein, e.g., a therapeutically effective amount of an HRD1 nucleic acid, polypeptide, or functional fragment thereof, or a therapeutically effective amount of a nucleic acid that inhibits IRE1 activity.

Also within the invention is an HRD1 nucleic acid, polypeptide, or functional fragment thereof for use in the treatment of an ER stress disorder, and an HRD1 nucleic acid, polypeptide, or functional fragment thereof use of in the manufacture of a medicament for the therapeutic and/or prophylactic treatment of an ER stress disorder.

The terms “RNA,” “RNA molecule,” and “ribonucleic acid molecule” refer to a polymer of ribonucleotides. The terms “DNA,” “DNA molecule,” and “deoxyribonucleic acid molecule” refer to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “snRNA” or “small nuclear RNA” is a single-stranded RNA precursor of mRNA. “mRNA” or “messenger RNA” is single-stranded RNA that specifies the amino acid sequence of one or more polypeptide chains. This information is translated during protein synthesis when ribosomes bind to the mRNA. The term “cDNA” or “complementary DNA” refers to a DNA molecule that has a sequence that is complementary to an mRNA or portion thereof, and can include single or double-stranded molecules, but is typically double-stranded.

The term “endoplasmic reticulum stress” (“ER stress”) refers to an imbalance between the demand that a load of proteins makes on the ER and the actual folding capacity of the ER to meet that demand. A response that counteracts ER stress has been termed “unfolded protein response” (“UPR”).

The term “ER stress disorder” refers to a disease or disorder (e.g., a human disease or disorder) caused by, or contributed to by, increased ER stress levels. Exemplary ER stress disorders include diabetes (e.g., type 1 or type 2 diabetes) and some protein conformational diseases. The term “protein conformational disease” (“PCD”) refers to a disease or disorder (e.g., a human disease or disorder) associated with protein misfolding (e.g., caused by, or contributed to by, protein misfolding). Exemplary protein conformational diseases include, but are not limited to, those diseases listed in Table 1. Other diseases include inflammatory bowel disease (Crohn disease and ulcerative colitis); and cancers originated from secretory cells (e.g., breast cancer and prostate cancer).

TABLE 1
Exemplary ER Stress Disorders/Protein Conformational Diseases

| Disease | Protein involved |
|---|---|
| Alzheimer's disease | amyloid-β |
| immunoglobulin light chain | immunoglobulin light chain |
| amyloidosis | |
| Parkinson's disease | alpha-synuclein |
| diabetes mellitus type 2 | amylin |
| amyotrophic lateral sclerosis (ALS) | Superoxide dismutase (SOD) |
| haemodialysis-related amyloidosis | L2-microglobulin |
| reactive amyloidosis | amyloid-A |
| cystic fibrosis | cystic fibrosis transmembrane |
| | regulator (CFTR) |
| sickle cell anemia | hemoglobin |
| Huntington's disease | huntingtin |
| Kreutzfeldt-Jakob disease and | prions (PrP) |
| related disorders (prion | |
| encephalopathies) | |
| familial hypercholesterolaemia | low density lipoprotein (LDL) |
| | receptor |
| Alpha1-antitrypsin deficiency, | Alpha1-antitrypsin (alpha1-AT) |
| cirrhosis, emphysema | |
| systemic and cerebral hereditary | (ten other proteins) |
| amyloidoses | |
| Wolcott-Rallison syndrome | translation initiation factor 2-alpha |
| | kinase-3 |
| Wolfram syndrome | Wolfram syndrome 1 (WFS1) |

Various methodologies described herein include steps that involve comparing a value, level, feature, characteristic, property, etc. to a “suitable control,” referred to interchangeably herein as an “appropriate control.” A “suitable control” or “appropriate control” can be any control, reference, or standard known to one of ordinary skill in the art that is useful for comparison purposes. In one embodiment, a “suitable control” or “appropriate control” is a value, level, feature, characteristic, property, etc. determined prior to performing a methodology of the invention described herein. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc. An “increase” or “decrease” can be determined relative to a suitable control.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram of unspliced and spliced mouse XBP-1 mRNAs. The coding regions are boxed, the bZip domain is shaded gray, and the 26-base pair nucleotide region processed by IRE1 is colored black. The active form of XBP-1 mRNA (cDNA) loses 26 base pairs by IRE1 processing. The spliced form of XBP-1 mRNA encodes a larger and active form of XBP-1 protein. The inactive form of XBP-1 cDNA is smaller than the DNA fragment of the active form of XBP-1.

FIG. 1B is a reproduction of a gel stained with ethidium bromide (EtBr) showing the results of RT-PCR analysis done with a primer set encompassing the splice junction of XBP-1 mRNA. PCR products were resolved on a 2.5% agarose gel to separate spliced (active form) and unspliced XBP-1 mRNAs. Wild-type or IRE1 mutant mouse embryonic fibroblast cells were untreated or treated with Tunicamycin (Tm) or Thapsigargin (Tg). Total RNA was prepared at the indicated times. The spliced (encoding active form of XBP-1) and unspliced (encoding inactive form of XBP-1) cDNA fragments are indicated by the arrows.

FIG. 2A is a schematic diagram of unspliced and spliced murine XBP-1 mRNAs. The coding regions are boxed, the bZip domain is shaded grey, and the 26-base pair nucleotide region processed by IRE1 is colored black. The active form of XBP-1 mRNA (cDNA) loses its Pst I site by IRE1 processing. The spliced form of XBP-1 mRNA encodes a larger, active form of XBP-1 protein. Thus, the inactive form of XBP-1 cDNA, when digested with Pst I, produces two DNA fragments that are smaller than the DNA fragment of the active form of XBP-1 produces when digested with Pst I.

FIG. 2B is a reproduction of a gel stained with ethidium bromide (EtBr) showing Pst I digested XBP-1 cDNA from wild-type or IRE1 mutant cells that were untreated or treated with Tunicamycin (TM) or Thapsigargin (Tg). Total RNA was prepared at the indicated times. The spliced (encoding an active form of XBP-1) and unspliced (encoding an inactive form of XBP-1) cDNA fragments are indicated by the arrows.

FIG. 3 is a reproduction of a gel stained with ethidium bromide (EtBr) showing Pst I digested XBP-1 cDNA from mouse islet cells that were untreated (Control) or treated with 1 mM of dithiothreitol (DTT) for 4 hours. The spliced (encoding active form of XBP-1) and unspliced (encoding inactive form of XBP-1) cDNA fragments are indicated by the arrows.

FIG. 4 is a reproduction of a gel stained with ethidium bromide (EtBr) showing XBP-1 splicing in MIN-6 cells expressing the insulin-2 gene with an Akita mutation. Pst I digested XBP-1 cDNA was isolated from MIN6 cells untransfected (Control), transfected with wild-type Insulin 2 expression vector (Ins2 WT) or with insulin-2 containing Akita mutation expression vector (Ins2 Akita).

FIGS. 5A and 5B are representations of the mRNA (5A, SEQ ID NO:1) and amino acid (5B, SEQ ID NO:2) sequences of the spliced form of XBP-1. The underlined regions of the mRNA sequence correspond to (or are reverse complements of) primers (SEQ ID NOs:8 and 9) for amplifying a region of the human XBP-1 mRNA that includes a splice junction. The splice junction is between nucleotides 506 and 507. The bold, underlined regions of the amino acid sequence is the sequence of the C-terminal portion of the protein encoded by the spliced form (SEQ ID NO: 6) that differs from that encoded by the unspliced form, which is bold and underlined in FIG. 6B.

FIGS. 6A and 6B are representations of the mRNA (6A, SEQ ID NO:3) and amino acid (6B, SEQ ID NO:4) sequences for the unspliced form of XBP-1. The underlined regions of the mRNA sequence correspond to (or are reverse complements of) primers (SEQ ID NOs:8 and 9) for amplifying a region of the human XBP-1 mRNA that includes a splice junction. The boxed region of the nucleotide sequence is the sequence spliced out by IRE1 (SEQ ID NO:5). The splice junction is between nucleotides 506 and 507 in FIG. 5A. The bold, underlined region of the amino acid sequence is the sequence of the C-terminal portion of the protein encoded by the unspliced form (SEQ ID NO:7) that differs from that encoded by the spliced form, which is bold and underlined in FIG. 5B.

FIG. 7 is a graph illustrating the standard curve for amplification of the spliced XBP-1 target detected using a cybergreen-labeled probe. Ct is the threshold cycle.

FIG. 8 is a graph illustrating the standard curve for amplification of the unspliced XBP-1 target detected using a cybergreen-labeled probe. Ct is the threshold cycle.

FIG. 9 is a Western blot analysis of wild-type and kinase inactive K599A (IRE1aKA) human IRE1a expressed in COS7 cells using PIRE1A1 antibody (P—IRE1a) or total IRE1a antibody. PIRE1A1 antibody specifically detects wild-type IRE1a, which is known to be autophosphorylated by over-expression.

FIG. 10 is a Western blot showing the effect of coexpression of ubiquitin^{K48R} on the expression level of wild-type or P724L WFS1. Lanes 1 and 3: COS7 cells transfected with wild-type or P724L WFS1 expression vector alone. Lanes-2 and 4: COS7 cells costransfected with HA-tagged ubiquitin^{K48R} (UbK48R) expression vector.

FIG. 11 is a Western blot showing the results of immunoprecipitation of ubiquitin immunoreactive polypeptides with anti-WFS1 antibody. Fibroblasts from an unaffected individual (control) and a patient with Wolfram syndrome (WFS) were lysed in detergent. Cells were treated (+) or untreated (−) with MG132 (2 mM) for 16 hours. Detergent-soluble fractions were immunoprecipitated by anti-WFS1 antibody, separated on 4-20% linear gradient SDS-PAGE and immunoblotted with anti-ubiquitin antibody.

FIG. 12 is a Western blot showing high-molecular-weight complexes of WFS1P724L in detergent-insoluble fractions. COS7 cells transfected with Flag-tagged wild-type or P724L WFS1 expression vector were separated into detergent-soluble (upper panel) and detergent-insoluble (lower panel) fractions and immunoblotted with anti-Flag antibody.

FIG. 13A is a Western blot showing ubiquitination of WFS1 by EDEM. COS7 cells were cotransfected with Flag-tagged wild-type or P724L mutant WFS1, Myc-tagged EDEM, and HA-tagged ubiquitin. Cells were lysed in detergent, immunoprecipitated with anti-Flag antibody, and immunoblotted with anti-HA antibody.

FIG. 13B is a Western blot showing the association of EDEM with mutant WFS1. COS7 cells were co-transfected with Flag-tagged wild-type or P724L WFS1 and Myc-tagged EDEM. Lysates were immunoprecipitated with anti-Flag antibody and immunoblotted with anti-Myc antibody.

FIG. 13C is a bar graph illustrating that EDEM is upregulated in lymphocytes from WFS patients. Quantitative real-time PCR of reverse transcribed RNA of lymphoblast cells from Wolfram syndrome patients (WFS), their relatives who are heterozygous for the WFS1 mutation (Hetero), and the relatives who are homozygous normal. The amount of EDEM mRNA was normalized to the amount of GAPDH mRNA in each sample (n=8, values are mean±s.e.m.)

FIGS. 14A-D are four bar graphs illustrating the results of quantitative real-time PCR of WFS1 using reverse-transcribed RNA from wild-type (WT) and Ire1α knock-out (Ire1α−/−) mouse embryonic fibroblast cells. Cells were untreated or treated with tunicamycin (TM) (14A and B), thapsigargin (TG) (13C) or dithiothreitol (DTT) (14D) for six hours. EDEM expression by TM was also shown as control (14B). The amount of mouse WFS1 and EDEM mRNA was normalized to the amount of GAPDH mRNA in each sample.

FIG. 15 is a bar graph illustrating the levels of expression of BiP, Hrd1, and Sel1L mRNA in the islets of Akita mice, as determined by quantitative real-time PCR of reverse-transcribed RNA from the islets of Akita mice (Ins2^{C96Y}/WT) and wild-type mice (WT/WT). The amount of transcript of the gene of interest was normalized to the amount of GAPDH RNA in each sample. The mean±SEM from six animals for each genotype is shown.

FIG. 16 is a bar graph showing the relative expression of the active form of XBP-1 mRNA in mouse embryonic fibroblast cells, as determined by quantitative real-time PCR of reverse-transcribed RNA from wild-type (WT) and Ire1α knock-out (Ire1a−/−) mouse embryonic fibroblast cells. Cells were treated or untreated with Tunicamycin (TM), an ER stress inducer, for two hours. The ratio of relative XBP-1 mRNA levels (spliced versus unspliced) is shown.

FIG. 17 is a bar graph showing the expression of the active form of XBP-1 mRNA in the islets of Akita mice, as determined by quantitative real-time PCR of reverse-transcribed RNA from the islets of Akita mice (Ins2^{C96Y}/WT) and wild-type mice (WT/WT). The ratio of relative XBP-1 mRNA levels (spliced versus unspliced) is shown. The mean±SEM from six animals for each genotype is shown.

FIG. 18 is a pair of Western blots showing the effect of proteasome inhibitor on the steady-state expression level of wild-type or C96Y insulin 2. Lanes 1 and 3: COS7 cells transfected with wild-type or C96Y insulin 2 expression vector alone. Lanes 2 and 4: COS7 cells transfected with Flag-tagged ubiquitin^{K48R} (Ub K48R) expression vector and treated with MG132 (20 mM). Actin was used as a loading control.

FIG. 19 is a pair of Western blots showing the effect of expression of ubiquitin^{K48R} on the expression level of wild-type or C96Y insulin 2. Lanes 1 and 3: COS7 cells transfected with wild-type or C96Y insulin 2 expression vector alone. Lanes 2 and 4: COS7 cells cotransfected with Flag-tagged ubiquitin^{K48R} (Ub K48R) expression vector. Actin was used as a loading control.

FIG. 20 is a Western blot showing the ubiquitination of insulin by HRD1. COS7 cells were transfected with expression vectors for HA-tagged wild-type or C96Y mutant insulin 2, HRD1, and Flag-tagged ubiquitin. Cells were lysed in detergent, immunoprecipitated with anti-HA antibody, and immunoblotted with anti-Flag antibody. Shown below are expression levels of insulin 2 and HRD1 input measured by immunoblot.

FIG. 21 is a model for the pathogenesis of Wolfram syndrome. The death of β cells in patients with Wolfram syndrome is a result of the combined effects of misfolded WFS1 proteins and the lack of functional WFS1 protein in cells.

FIG. 22A is a pair of immunoblots showing the results of analysis of phospho Ire1α (P-IRE1α) using lysates from mouse islets and whole pancreas. Actin was used as a loading control.

FIG. 22B is a pair of immunoblots showing the results of analysis of P-IRE1α (P-IRE1α) using lysates from different cell lines. Actin was used as a loading control.

FIG. 23A is a series of immunoblots of MIN6 cells treated with 5 mM or 25 mM glucose; Phospho-Ire1α (P-Ire1a), was detected by immunoblot analysis with anti-phospho specific IRE1a antibody. Also detected were cellular expression levels of total Ire1α, insulin, protein disulfide isomerase (Pdi), and actin by immunoblot analysis using the same lysates, and levels of insulin secreted into the media. Actin was used as a loading control.

FIG. 23B is a trio of immunoblots of P-Ire1α and insulin in INS1 cells treated with 0 mM, 2.5 mM, 10 mM, 20 mM, and 25 mM of glucose. Actin was used as a loading control.

FIG. 24A is a trio of immunoblots of total Ire1α and insulin in MIN6 cells treated with siRNA specific for Ire1α. Actin was used as a loading control.

FIG. 24B is a trio of immunoblots of total Ire1α and insulin in INS1 cells treated with siRNA specific for Ire1α. Actin was used as a loading control.

FIG. 25 is a model for the relationship between physiological ER stress and insulin biosynthesis.

FIGS. 26A and 26B are representations of the mRNA (26A, SEQ ID NO:38) and amino acid (26B, SEQ ID NO:39) sequences for the a isoform of human HRD1 (Genbank Accession No. NM_{—}032431, protein ID NP_{—}115807.1). The bold region of the amino acid sequence is the RING domain.

FIGS. 27A and 27B are representations of the mRNA (27A, SEQ ID NO:40) and amino acid (27B, SEQ ID NO:41) sequences for the b isoform of human HRD1 (Genbank Accession No. NM_{—}172230, protein ID NP_{—}757385.1). The bold region of the amino acid sequence is the RING domain.

### DETAILED DESCRIPTION

Since the ER stress signaling network plays a role in the pathogenesis of many human diseases, it is important to monitor the ER stress level in mammalian cells. The present invention includes methods and reagents to quantify ER stress levels, and methods and compositions for treating and diagnosing ER stress disorders.

IRE1 is an upstream component of the ER stress signaling network and it is a sensor for ER stress. Some of the methods described herein feature quantifying IRE1 activity levels as a measure of ER stress. Because it can be difficult to measure IRE1 activity levels directly, XBP-1 mRNA splicing levels, which precisely reflect IRE1 activation, can be used to quantify the IRE1 activity levels. Spliced XBP-1 mRNA encodes the active XBP-1 transcription factor and activates the UPR. The invention features methods to quantify the activity level of XBP-1 using Reverse Transcriptase-PCR (RT-PCR). Primers are designed to amplify the region encompassing the splice junction of XBP-1 mRNA. The spliced (active) form of XBP-1 mRNA (cDNA) is smaller than the unspliced (inactive) form by 26 base pairs. The size difference between the two forms can be visualized, for example, by electrophoresing the PCR products on an agarose gel.

Various aspects of the invention are described in further detail in the following subsections.

I. ER Stress and ER Stress Signaling Pathway Assays

The unfolded protein response (UPR) is a cellular adaptive response that counteracts ER stress. The UPR includes three different pathways to address ER stress: (1) gene expression, (2) translational attenuation, and (3) protein degradation. Inositol Requiring 1 (IRE1), an ER-resident transmembrane protein kinase, is one of the furthest upstream components of the UPR, and acts as a central regulator for UPR-specific downstream gene expression and apoptosis. At least in part, IRE1 acts by splicing a small intron from XBP-1 mRNA.

IRE1 and XBP-1 are crucial components of the UPR, and the expression levels of the active forms of XBP-1 and IRE1 can serve as markers for ER stress levels. It is difficult to directly measure the activity level of IRE1, because although activation of IRE1 by phosphorylation causes a shift to lower mobility on an SDS-polyacrylamide gel, the shift is very small and thus difficult to detect. To overcome this difficulty, some of the new methods described herein use XBP-1 splicing as a measure of ER stress level.

XBP-1 mRNA splicing levels can be detected using any method known in the art, e.g., Northern blotting, in situ hybridization (Parker and Barnes, Methods in Molecular Biology 106:247-283 (1999)), RNAse protection assays (Hod, BioTechniques 13:852-854 (1992); Saccomanno et al., BioTechniques 13: 846-85 (1992)), or reverse transcription polymerase chain reaction (RT-PCR) (Weis et al., Trends in Genetics 8:263-264 (1992)).

In some embodiments, splice levels are detected using a nucleic acid probe, e.g., a labeled probe (a number of suitable labels are known in the art, including radioactive, fluorescent, spin, and calorimetric labels), that hybridizes to the intron that is removed from the XBP-1 sequence by splicing.

In some embodiments, XBP-1 splicing is detected using RT-PCR (reverse transcription-polymerase chain reaction, typically involving cDNA synthesis from a target mRNA by reverse transcription, followed by PCR amplification) and a pair of primers designed to amplify a region including the splice site. RT PCR methods are known in the art.

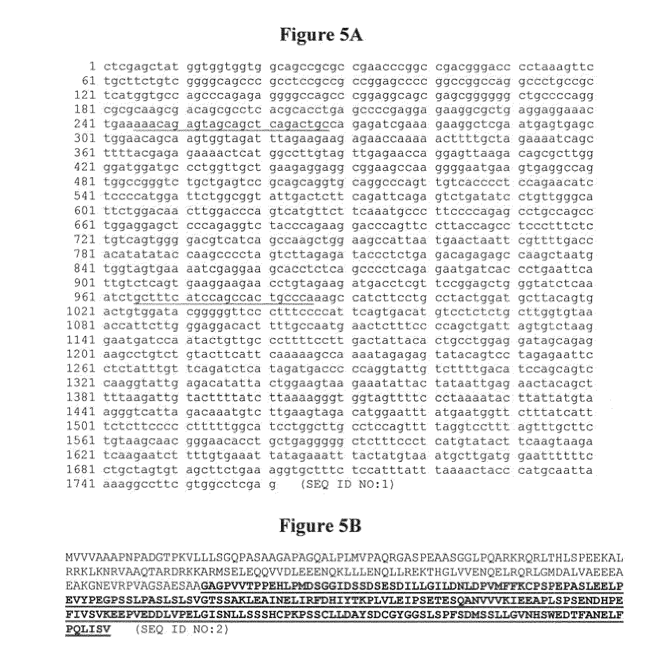

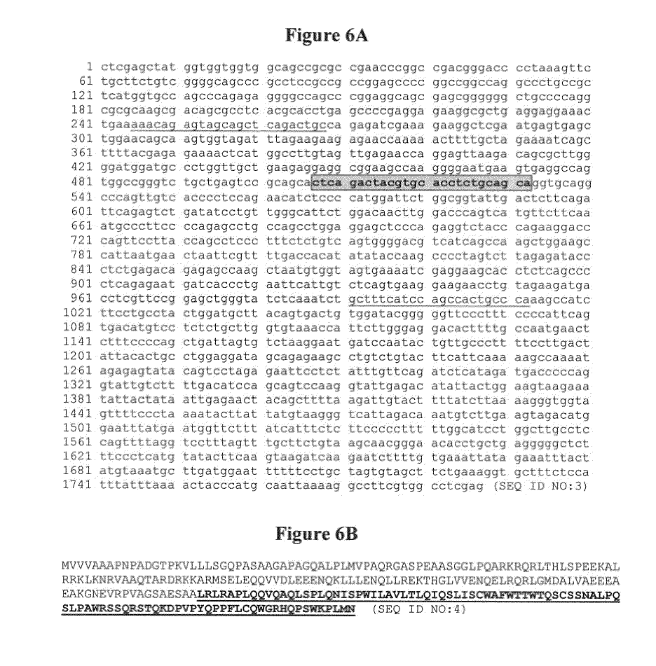

In some embodiments, the methods described herein measure splicing of XBP-1 by RT-PCR, optionally followed by Pst I digestion (See Examples 2-4). The mRNA and amino acid sequences for the spliced and unspliced forms of XBP-1 are shown in FIGS. 5A and B and 6A and B, respectively. The underlined regions of each sequence correspond to (or are reverse complements of) primers for amplifying a region of the human XBP-1 mRNA that includes a splice junction. Additional primer pairs can readily be designed by the skilled artisan given the above sequences and primer design programs. The boxed region of the nucleotide sequence in FIG. 6A is the sequence spliced out by IRE1. The splice junction is between nucleotides 506 and 507 in FIG. 5A. The bold, underlined regions of the amino acid sequence in FIG. 5B is the sequence of the protein encoded by the spliced form that differs from that encoded by the unspliced form, which is bold and underlined in FIG. 6B.

In some embodiments, real-time PCR, e.g., as described in Bustin et al., J. Mol. Endocrinol. 25:169-193 (2000), is used, for example, when more accurate quantification of splicing levels is required, e.g., where splicing levels are neither very high (e.g., most of the XBP-1 is in spliced form) nor very low (e.g., only some of the XBP-1 is in spliced form), but are in between (e.g., there is a more nearly balanced mixture of spliced and non-spliced XBP-1).

As noted above, any pairs of primers that can amplify the region of the target XBP-1 mRNA that includes a splice junction can be used. Exemplary sequences for primers are provided herein. Typically, the primer set will include a first primer that is identical to or complementary to a sequence that is 5′ of the spliced intron region, and a second primer that is identical to or complementary to a sequence that is 3′ of the spliced intron region, such that when the two primers are used in a polymerase chain reaction, a region of suitable size is obtained. One of skill in the art will be able to design a suitable set of primers using the sequences of XBP-1 known in the art and provided herein.

In some embodiments, levels of ER stress are detected using a binding agent specific for the spliced or unspliced form of XBP-1 protein. In some embodiments, the binding agent is an antibody that is specific for the spliced or unspliced form, e.g., recognizes an epitope that is 3′ of the splice site. For example, an antibody that is specific for the spliced form can recognize an epitope in SEQ ID NO:6; an antibody specific for the unspliced form can recognize an epitope in SEQ ID NO:7. Such antibodies can include any form-specific antibody (e.g., a monospecific, or a recombinant or modified antibody), and includes antigen-binding fragments thereof (e.g., Fab, F(ab′)₂, Fv or single chain Fv fragments).

In some embodiments, levels of ER stress are detected using a binding agent specific for the auto-phosphorylated form of IRE1α, e.g., an antibody that specifically binds to the auto-phosphorylated form, but does not substantially bind to the non-phosphorylated form.

The antibodies can be of the various isotypes, including: IgG (e.g., IgG₁, IgG₂, IgG₃, IgG₄), IgM, IgA₁, IgA₂, IgD, or IgE. The antibody molecules can be full-length (e.g., an IgG₁ or IgG₄ antibody) or can include only an antigen-binding fragment (e.g., a Fab, F(ab′)₂, Fv or a single chain Fv fragment). These include monoclonal antibodies, recombinant antibodies, chimeric antibodies, humanized antibodies, deimmunized antibodies, as well as antigen-binding fragments of the foregoing.

Antibodies (e.g., monoclonal antibodies from differing organisms, e.g., rodent, sheep, human) can be produced using art-recognized methods. Once the antibodies are obtained, the variable regions can be sequenced. The location of the CDRs and framework residues can be determined (see, Kabat et al., Sequences of Proteins of Immunological Interest, Fifth Edition, U.S. Department of Health and Human Services, NIH Publication No. 91-3242 (1991), and Chothia et al., J. Mol. Biol. 196:901-917 (1987)). The light and heavy chain variable regions can, optionally, be ligated to corresponding constant regions. Light and heavy immunoglobulin chains can be generated and co-expressed into the appropriate host cells.

Monoclonal antibodies can be used in the methods described herein. Monoclonal antibodies can be produced by a variety of known techniques, including conventional monoclonal antibody methodology e.g., the standard somatic cell hybridization technique of Kohler and Milstein, Nature 256: 495 (1975). See generally, Harlow and Lane, Using Antibodies: A Laboratory Manual, Cold Spring Harbor Laboratory Press, Cold Spring Harbor, N.Y. (1999). Although somatic cell hybridization procedures can be used, as well as other techniques for producing monoclonal antibodies, e.g., viral or oncogenic transformation of B lymphocytes. A typical animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying human immunoglobulin genes rather than corresponding mouse genes. Splenocytes from these transgenic mice immunized with the antigen of interest are used to produce hybridomas that secrete human mAbs with specific affinities for epitopes from a human protein (see, e.g., Wood et al., International Application WO 91/00906, Kucherlapati et al., PCT publication WO 91/10741; Lonberg et al., International Application WO 92/03918; Kay et al., PCT publication WO 92/03917; Lonberg et al., Nature 368:856-859 (1994); Green et al., Nature Genet. 7:13-21 (1994); Morrison et al., Proc. Natl. Acad. Sci. USA 81:6851-6855 (1994); Bruggeman et al., Year Immunol. 7:33-40 (1993); Tuaillon et al., Proc. Natl. Acad. Sci. USA 90:3720-3724 (1993); Bruggeman et al., Eur. J. Immunol. 21:1323-1326 (1991). Thus, the invention includes antibodies specific for a spliced or unspliced form of XBP-1, and for the autophosphorylated form of IRE1.

Useful immunogens for the purpose of producing anti-XBP-1 antibodies include peptides comprising portions of XBP-1 that are unique to either the spliced or unspliced form of XBP-1, e.g., all or part of the sequences shown in SEQ ID NOs:6 (spliced form) and 7 (unspliced form). Useful immunogens for the purpose of producing antibodies specific for the autophosphorylated form of IRE1 include phosphopeptides comprising the sequence surrounding the autophosphorylation site, wherein the autophosphorylation site is phosphorylated (e.g., see Example 6).

The antibodies can be labeled to facilitate detection and quantification of XBP-1 splicing or IRE1 autophosphorylation levels. Numerous suitable labels, and methods for labeling the antibodies, are known in the art. Examples of suitable labels include a fluorescent label, a biologically active enzyme label, a radioisotope (e.g., a radioactive ion), a nuclear magnetic resonance active label, a luminescent label, or a chromophore. In some embodiments, a labeled secondary antibody is used. See, e.g., Harlow and Lane, supra.

Quantitation can be performed using any method known in the art, including but not limited to fluorometry, gamma counting, scintillation counting, spectrophotometry, kinetic phosphorescence, or phosphorimaging. Computer-based methods can be used to facilitate analysis.

In some embodiments, quantitation of ER stress is performed in the cells or tissues directly affected by a selected condition, e.g., neural tissue in the case of neurodegenerative disease, or islet cells in the case of diabetes and related disorders. In other embodiments, quantitation of ER stress is performed in another cell type, e.g., peripheral blood cells such as lymphocytes. As described herein, lymphocytes from individuals suffering from WFS have elevated levels of ER stress as compared to normal controls, and thus are a useful proxy for detecting elevated ER stress levels in situations, such as with human subjects, when using the affected cell type is impractical or otherwise undesirable.

II. Treatment and Diagnosis of ER Stress Disorders, and Methods of Screening

Mutations in integral membrane proteins, such as the cystic fibrosis transmembrane conductance regulator protein, are known to cause the accumulation of misfolded proteins in the ER, which, in turn, causes a particular type of intracellular stress termed ER stress (Harding et al., Annu. Rev. Cell. Dev. Biol. 18:575-599 (2002)). Accumulating evidence suggests that a high level of ER stress or defective ER stress signaling causes β-cell death in the development of diabetes (Harding and Ron, Diabetes 51(Suppl 3):S455-461 (2002)). The unfolded protein response (UPR) is an intracellular stress management system that counteracts ER stress (Harding et al., Annu. Rev. Cell. Dev. Biol. 18:575-599 (2002); Kaufman et al., Nat. Rev. Mol. Cell. Biol. 3:411-421 (2002); Mori, Cell 101:451-454 (2000)). The UPR has three components: gene expression, translational attenuation, and ER-associated protein degradation (the ERAD system). The ERAD system has an important function in the survival of ER stressed cells. The methods are discussed herein using Wolfram Syndrome (a protein conformational disease) and diabetes mellitus (an ER stress disorder that may not be a protein conformational disease) as examples, but the results can be extrapolated to other ER stress disorders.

Wolfram Syndrome

Wolfram syndrome (WFS) is a rare form of juvenile diabetes in which pancreatic β-cell death is not accompanied by an autoimmune response. Wolfram syndrome was first reported in 1938 by Wolfram and Wagener (Wolfram and Wagener, Mayo Clin. Proc. 1:715-718 (1938)), who analyzed four siblings with the combination of juvenile diabetes and optic atrophy. Because a significant portion of patients with Wolfram syndrome develop diabetes insipidus and auditory nerve deafness, this syndrome is also referred to as the diabetes insipidus, diabetes mellitus, optic atrophy, and deafness (DIDMOAD) syndrome (Barrett and Bundey, J. Med. Genet. 34:838-841 (1997); Rando et al., Neurology 42:1220-1224 (1992)). Its pathogenesis is still unknown. Patients with Wolfram syndrome do not have either insulitis or obesity. However, β cells in pancreatic islets are selectively destroyed (Karasik et al., Diabetes Care 12:135-138 (1989)). The mechanism of (3-cell death seen in Wolfram syndrome patients may be the same as, or similar to, the accelerated form of cell death seen in type-2 diabetes patients. Families that exhibit Wolfram syndrome share mutations in a gene encoding WFS1 protein, a trans-membrane protein in the endoplasmic reticulum (ER) (Inoue et al., Nature Genetics 20:143-148 (1998); Strom et al., Hum. Mol. Genet. 7:2021-2028 (1998)). Most of the WFS1 mutations in Wolfram syndrome patients occur in exon 8, including the P724L mutation.

As described herein (see Examples 7-10), the mutant WFS1 protein seen in patients with Wolfram syndrome accumulates in the ER and activates its associated system for degrading mutant proteins in the endoplasmic reticulum. In lymphoblast cells from patients with Wolfram syndrome, expression of endoplasmic reticulum degradation-enhancing alpha-mannosidase-like protein, a central component of the protein degradation system, is significantly upregulated. In addition, we show that mutant WFS1 protein tends to form insoluble aggregates that are not degraded by this system.

The results described herein indicate that the pathogenesis of Wolfram syndrome can be attributed to the combined effects of a lack of functional WFS1 protein and the presence of insoluble WFS1 aggregates in cells (FIG. 21). Thus, the methods described herein can be used to identify new clinical approaches, based on the prevention of β-cell death by therapeutic agents that will block the ER stress-mediated cell-death pathway, for the treatment of Wolfram Syndrome.

Diabetes Mellitus

Pancreatic β-cell death contributes to both type 1 and type 2 diabetes. More than one million people suffer from type 1 diabetes in the U.S. In this disease, insulin production is abnormally low due to the destruction of beta cells in pancreatic islets. Chronic ER stress in β cells is likely to play a role in the pathogenesis of diabetes; recent observations in the Akita diabetes model mouse (a C57BL/6 mouse with a mutation in insulin 2 gene) support the hypothesis that sufficient endoplasmic reticulum (ER) stress can cause beta-cell death, see Harding and Ron, Diabetes 51(Suppl 3):S455-461 (2002) Oyadomari et al., J. Clin. Inv. 109:525-32 (2002); and Urano et al., Science 287:664-6 (2000). A diagnosis of type 1 diabetes mellitus can be made, e.g., on the basis of symptom history confirmed by a blood or plasma glucose level greater than 200 mg/dl, with the presence of glucosuria and/or ketonuria. Other symptoms representative of autoimmune diabetes are polyuria, polydipsia, weight loss with normal or even increased food intake, fatigue, and blurred vision, commonly present 4 to 12 weeks before the symptoms are noticed. Before clinical onset of type 1 diabetes mellitus, diagnosis may be possible with serologic methods, e.g., complemented by beta cell function tests.

A positive effect on a parameter associated with diabetes can be one or more of the following: (1) decreasing plasma glucose levels and urine glucose excretion to eliminate polyuria, polydipsia, polyphagia, caloric loss, and adverse effects such as blurred vision from lens swelling and susceptibility to infection, particularly vaginitis in women, (2) abolishing ketosis, (3) inducing positive nitrogen balance to restore lean body mass and physical capability and to maintain normal growth, development, and life functioning, and (4) preventing or greatly minimizing the late complications of diabetes, i.e., retinopathy with potential loss of vision, nephropathy leading to end stage renal disease (ESRD), and neuropathy with risk of foot ulcers, amputation, Charcot joints, sexual dysfunction, potentially disabling dysfunction of the stomach, bowel, and bladder, atherosclerotic cardiovascular, peripheral vascular, and cerebrovascular disease. A negative effect on a parameter would be the opposite of these four factors. The current American Diabetes Association standards of care include (1) maintaining preprandial capillary whole blood glucose levels at 80 to 120 mg/di, bedtime blood glucose levels at 100 to 140 mg/dl, and postprandial peak blood glucose levels at less than 180 mg/dl, and (2) maintaining an HbA1c of less than 7.0% (relative to a non-diabetic DCCT range of approximately 4.0% to 6.0%).

Treatment and Diagnosis of ER Stress Disorders

Quantifying or detecting ER stress is useful in any situation where it is suspected or has been determined that such stress may regulate a normal cellular phenotype (e.g., regulate apoptosis) or cause or contribute to a disease phenotype (e.g., a protein conformational disease phenotype such as Wolfram Syndrome or diabetes). In mammalian cells, ER stress is regulated, at least in part, by an ER stress signaling pathway. This pathway is an evolutionarily conserved signaling network that is emerging as the major quality controller of newly synthesized proteins.

ER stress signaling is likely to be crucial for protein secretion and the development of secretory cells, such as plasma cells, adipocytes, and trophoblast cells in placenta. The data described herein indicate that defects in this signaling network can cause or contribute to human diseases, such as the diseases listed in Table 1, as well as others, including some forms of juvenile diabetes, inflammatory bowel disease, and cancers originated from secretory cells (e.g., breast cancer and prostate cancer).

As it is believed that defects in the ER stress signaling network cause or contribute to human diseases including many of the diseases listed in Table 1, as well as others, as described herein, it is contemplated that the ER stress measurement methodologies described herein will be useful in methods for diagnosing any of these diseases in subjects. In some embodiments, the methods and reagents described herein can be used to diagnose the stage of a disease in patients. In some embodiments, the disease is multiple myeloma. As multiple myeloma is a cancer of plasma cells, and ER stress signaling is important for the development of plasma cells, it is expected that ER stress levels will be very high in multiple myeloma cells. Higher stress levels are likely to correlate to more aggressive disease.

The methods and reagents described herein are suitable for use in methods to further study the role of ER stress in cellular processes such as apoptosis and contribution of such processes in a variety of ER stress disorders, and in methods of screening for compounds, e.g., drugs, useful in the treatment of such diseases. Thus, in some embodiments, the methods include providing a test system, e.g., an ER stress model system, e.g., a cell or animal model of an ER stress disorder; optionally increasing levels of ER stress in the cells or animal (e.g., in at least some of the cells of the animal); contacting the cells with a test compound; and evaluating the levels of XBP-1 splicing in the cells in the presence and absence of the test compound, thus evaluating the effect of the compound on ER stress. Those compounds that produce a desired effect on ER stress, e.g., that significantly reduce ER stress (i.e., as measured by XBP-1 splicing levels), can be considered as candidate compounds and further evaluated for therapeutic activity using methods known in the art, e.g., administering the candidate compounds to an animal, e.g., an animal model of an ER stress disorder, and evaluating an effect of the compound on the animal, e.g., therapeutic efficacy or toxicity. In some embodiments, ER stress is reduced by at least about 20%, e.g., about 30%, 40%, 50%, 60%, 70%, 80%, 90%, or 100%.

In some embodiments, the methods described herein can be used to determine if a disease has an ER stress-related component, e.g., has an etiology that is due in part to ER stress. Such diseases can include cellular degenerative diseases such as neurodegenerative conditions. These methods can include, for example, determining levels of ER stress by a method described herein in a model system such as an animal or cellular model of the disease, or in cells from a human or animal having the disease. This information can be used to determine whether a subject suffering from a particular disease would benefit from the administration of an agent that decreases ER stress.

In some embodiments, the system is an animal model of an ER stress disorder, e.g., an ER stress disorder as described herein, or cells derived therefrom. Typically, an ER stress disorder can be induced in an otherwise healthy animal or cell by administering a compound known to cause ER dysfunction, e.g., by administering a sublethal dose of thapsigargin, tunicamycin (e.g., 0.25-1 mg/kg tunicamycin; see Zinszner et al., Genes and Dev. 12:982-995 (1998)), or a proteosome inhibitor, e.g., lactacystin. In some embodiments, the system is a model of a neurodegenerative disease.

Inhibitory Nucleic Acids

The therapeutic methods described herein can include the administration of compounds that include nucleic acid molecules that inhibit the expression or activity of a target gene related to ER stress, such as IRE1 or HRD1. These include antisense, siRNA, ribozymes, and other modified nucleic acid molecules such as PNAs.

RNA Interference

RNAi is a remarkably efficient process whereby double-stranded RNA (dsRNA, also referred to herein as si RNAs or ds siRNAs, for double-stranded small interfering RNAs,) induces the sequence-specific degradation of homologous mRNA in animals and plant cells (Hutvagner and Zamore, Curr. Opin. Genet. Dev.:12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell. 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002).)
Accordingly, the invention includes such molecules that are targeted to an HRD1, IRE1α or IRE1β RNA.

siRNA Molecules

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. The dsRNA molecules of the invention can be chemically synthesized, or can transcribed be in vitro from a DNA template, or in vivo from, e.g., shRNA. The dsRNA molecules can be designed using any method known in the art. Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acid compositions of the invention include both siRNA and modified siRNA derivatives, e.g., siRNAs modified to alter a property such as the pharmacokinetics of the composition, for example, to increase half-life in the body, e.g., crosslinked siRNAs. Thus, the invention includes siRNA derivatives that include siRNA having two complementary strands of nucleic acid, such that the two strands are crosslinked. In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying SiRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

dsRNA molecules targeting IRE1 can comprise the sequences of SEQ ID NOs:35, 36, or 37 as one of their strands, and allelic variants thereof:

siRNA Delivery

Synthetic siRNAs can be delivered into cells, e.g., by cationic liposome transfection and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

Animal cells express a range of noncoding RNAs of approximately 22 nucleotides termed micro RNA (miRNAs) and can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra). Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Nanoparticles and liposomes can also be used to deliver siRNA into animals.

Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism.

Antisense

An “antisense” nucleic acid can include a nucleotide sequence that is complementary to a “sense” nucleic acid encoding a protein, e.g., complementary to the coding strand of a double-stranded cDNA molecule or complementary to a TEF mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence encoding the target gene (e.g., the 5′ and 3′ untranslated regions).

An antisense nucleic acid can be designed such that it is complementary to the entire coding region of a target mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a selected protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual n-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to the regulatory region of the target gene (e.g., promoters and/or enhancers) to form triple helical structures that prevent transcription of the Spt5 gene in target cells. See generally, Helene, C. Anticancer Drug Des. 6:569-84 (1991); Helene, C. Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target nucleic acid can include one or more sequences complementary to the nucleotide sequence of a target cDNA disclosed herein, and a sequence having known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a target mRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel, D. and Szostak, J. W. Science 261:1411-1418 (1993).

Methods of Screening

The methods described herein can be used in screening methods, e.g., high-throughput screening methods, e.g., to screen a library of test compounds, e.g., to identify candidate therapeutic agents for use in the treatment of an ER stress disorder as described herein. For example, antibody-based, fluorescence-based, or PCR-based high-throughput screening methods are known in the art and can be used to detect an effect on ER stress levels, e.g., by measuring IRE1 activity levels, for example, by measuring XBP-I splicing levels or IRE1 autophosphorylation levels.

For example, the methods described herein can be used to identify compounds and agents that modulate IRE1 activity and/or expression. IRE1 is a target for controlling insulin synthesis; a compound that increases IRE1 activity (e.g., a nucleic acid, a peptide, or a small molecule that increases IRE1 expression or IRE1 phosphorylation) is useful where increased insulin production is desired; a compound that decreases IRE1 activity (e.g., a nucleic acid such as an siRNA, ribozyme, morpholino oligo or antisense molecule, a peptide, or a small molecule that decreases IRE1 expression or IRE1 phosphorylation) is useful where decreased insulin production is desired. These compounds can be used to treat, e.g., diabetes or other insulin-related ER stress disorders. For example, compounds that decrease IRE1 activity can be used to regulate insulin production to treat hyperglycemia, a condition in which increased glucose leads to increased insulin biosynthesis. The increased load of misfolded insulin is believed to overload the ER stress response system, resulting in the death of the insulin-producing beta cells that leads to diabetes. Regulating the level of insulin produced can prevent the progression from hyperglycemia to diabetes.

As one example, illustrated in Example 5, an XBP-I/GFP fusion protein can be used to detect splicing levels; GFP (or any other detectable, e.g., fluorescent or chromatogenic, peptide or polypeptide) is cloned at the C-terminal end of XBP-I lacking a stop codon, in-frame with a spliced from of XBP-I. Since the splicing shifts the frame of the C-terminal portion of the protein, an active form of GFP will be produced only when spliced XBP-I is produced. This is a particularly useful measure of splicing as the ratio of GFP molecules will be about 1:1 with spliced XBP-I molecules, and detecting the GFP signal directly measures the amount of spliced XBP-I.

As another example, an antibody, e.g., an antibody described herein, that binds specifically to the autophosphorylated form of IRE1 can be used to determine levels of ER stress by detecting levels of IRE1 autophosphorylation. A number of methods are known in the art for using antibodies in this fashion.

High throughput methods for detecting fluorescence in cells are known in the art, and a number of commercially available systems can be adapted for use, e.g., systems using microplate readers, including those developed and used by Aventis, Genetix, Acumen, and Millipore. For example, for high throughput screens, multi-well plates, e.g., plates with 96, 384, or more separate areas, e.g., wells, e.g., separated by a barrier, can be screened. Suitable plates are known in the art, and can be manufactured, modified, or are commercially available. In some embodiments, each area, e.g., each well, contains a unique compound, e.g., small molecule of known or unknown structure, or a pool of molecules of known or unknown structure.

The test compound library can be a library of compounds of related or unrelated structures. Such libraries are known in the art and are commercially available or can be synthesized using methods known in the art.

Libraries of test compounds, such as small molecules, are available, e.g., commercially available, or can be synthesized using methods known in the art. As used herein, “small molecules” refers to small organic or inorganic molecules. In some embodiments, small molecules useful for the invention have a molecular weight of less than 10,000 Daltons (Da). The compounds can include organic or inorganic naturally occurring or synthetic molecules including, but not limited to, soluble biomolecules such as oligonucleotides, polypeptides, polysaccharides, antibodies, fatty acids, etc.

The compounds can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecule compounds are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1(1):60-66 (1997)). In addition, a number of compound, e.g., small molecule, libraries are commercially available.

Libraries and test compounds screened using the methods described herein can comprise a variety of types of compounds. A given library, for example, can comprise a set of structurally related or unrelated test compounds. In some embodiments, the compounds and libraries thereof can be obtained by systematically altering the structure of a first compound, e.g., a small molecule, e.g., using methods known in the art or the methods descried herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a test compound or compounds, e.g., a small molecule. For example, in one embodiment, a general library of small molecules is screened using the methods described herein.

In some embodiments, each well contains one or more unique text compounds, e.g., small molecules that are different from the test compounds in at least one of the other wells. In some embodiments, the multi-well plate also includes one or more positive and/or negative control wells. Negative control wells can contain, for example, no test compound other negative control. Positive control wells can contain, for example, compounds known to inhibit ER stress. In some embodiments, a number of multi-well plates, each comprising a unique set of small molecules, are screened. In this way, a library of test compounds in the hundreds, thousands, or millions can be screened for identification of ER stress reducing molecules.

Compounds identified as “hits” (e.g., compounds that decrease ER stress) in the first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using the methods described herein, identifying one or more hits in that library, subjecting those hits to systematic structural alteration to create additional libraries of compounds structurally related to the hit, and screening the second library using the methods described herein.

A test compound that has been screened by a method described herein and determined to have a desired activity (e.g., reduction of ER stress and/or increased levels of HRD1 (HMG-CoA reductase degradation) activity), can be considered a candidate compound. A candidate compound that has been screened, e.g., in an in vivo model of a disorder, e.g., an ER stress disorder, and determined to have a desirable effect on the disorder, e.g., on one or more symptoms of the disorder, can be considered a candidate therapeutic agent. Candidate therapeutic agents, once further screened, e.g., in a clinical setting, are therapeutic agents. Candidate therapeutic agents and therapeutic agents can be optionally optimized and/or derivatized, and formulated with pharmaceutically acceptable excipients to form pharmaceutical compositions.

The methods described herein are also suitable for use in methods of diagnosing ER stress disorders, e.g., as described herein, by evaluating ER stress levels in a subject, e.g., in a sample from a subject, e.g., a sample comprising cells such as peripheral blood cells, e.g., lymphocytes. For example, the methods and reagents can be used for diagnosing diabetes, e.g., Type 2 diabetes or certain forms of Type 1 diabetes, Wolcott-Rallison syndrome and Wolfram syndrome, as these diseases are believed to be caused, at least in part, by increased ER stress.

III. HRD1—A Novel Therapeutic Target for the Treatment of ER Stress Disorders

As noted above, both diabetes and WFS are characterized by loss of p cells. As described herein (see Example 11), HRD1 (hydroxymethylglutaryl reductase degradation 1), a component of the ERAD system, is upregulated in pancreatic islets of the Akita diabetes mouse model and enhances intracellular degradation of misfolded insulin. HRD1 is an E3 ligase, a key enzyme in the ubiquitination process. E3 ligases recognize protein substrates and facilitate the coupling of ubiquitin to the substrates, tagging them for degradation. High ER stress in β cells stimulates mutant insulin degradation through HRD1 to protect β cells from ER stress and ensuing death. The results described herein indicate not only that HRD1 is upregulated in the diabetes mouse model, but that HRD1 may be central to the protection of β cells from ER stress-mediated death. Thus, therapeutic agents that increase HRD1 levels and/or activity can be used to treat ER stress disorders. The methods described herein can be used to identify agents, such as peptides or small molecules, that activate or enhance the HRD1-mediated ERAD pathway, and may be therapeutically beneficial to patients with, or at risk for developing, ER stress disorders such as diabetes. These agents can be incorporated into pharmaceutical compositions for administration by an appropriate route.

In some embodiments, the methods described herein include determining the level of HRD1 expression or activity, e.g., using antibody-based detection, for example. A number of methods are known in the art for determining levels of expression or activity of a selected gene or protein (see, e.g., Kikkert et al., J. Biol. Chem. 279(5):3524-34 (2004); Deak and Wolf, J. Biol. Chem. 14(6):10663-10669 (2001)). The methods can further include determining whether a test compound has an effect on levels of HRD1 expression or activity, e.g., in a cell, or an animal. Test compounds that increase levels of HRD1 can be used to treat or prevent diabetes in subjects with high levels of ER stress, e.g., high levels of ER stress in the pancreatic islet cells, or in the lymphocytes. In some embodiments, test compounds that increase levels of HRD1 can be used to treat other ER stress disorders such as protein conformational diseases, and neurodegenerative diseases.

An E3 ligase, e.g., an HRD1-encoding nucleic acid, polypeptide, or a functional fragment thereof, can be administered to a person having an ER stress disorder such as diabetes, to thereby treat the ER stress disorder. A “functional fragment” of HRD1 is a fragment that retains at least 30% of the E3 ligase activity of the full-length HRD1 polypeptide, and includes at least one RING finger domain, e.g., amino acids 289-332 of the human HRD1 (e.g., Genbank Acc. No. NP_{—}115807 (SEQ ID NO:39) or NP_{—}757385 (SEQ ID NO:41)) or amino acids 208-551 of the yeast HRD1 (e.g., Genbank Acc. No. NP_{—}014630 or S66695), or a homologous region thereof. In some embodiments, an E1 ubiquitin-activating enzyme and/or an E2 ubiquitin-conjugating enzyme is administered in addition to the E3 ligase (e.g., HRD1). See, e.g., Kikkert et al., J. Biol. Chem. 279(5):3524-34 (2004); Deak and Wolf, J. Biol. Chem. 14(6):10663-10669 (2001). The HRD1 can be administered as part of a pharmaceutical composition, as described herein. In some embodiments, the methods described herein can be used to determine whether an HRD1 nucleic acid, polypeptide, or active fragment thereof is effective to treat a selected ER stress disorder, e.g., diabetes. For example, HRD1 is administered to a model, such as a cell or animal model, of the selected disease, and the model is monitored to determine whether the HRD1 has an effect on the model.

IV. Pharmaceutical Compositions and Methods of Administration

The therapeutic agents described herein can be incorporated into pharmaceutical compositions. Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with their intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

For administration by inhalation, the compounds are typically delivered in the form of an aerosol spray from pressured container or dispenser which contains a suitable propellant, e.g., a gas such as carbon dioxide, or a nebulizer. Such methods include those described in U.S. Pat. No. 6,468,798.

The therapeutic compounds can also be prepared in the form of suppositories (e.g., with conventional suppository bases such as cocoa butter and other glycerides) or retention enemas for rectal delivery.

Therapeutic compounds comprising nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996). In some embodiments, targeted delivery of a composition comprising a nucleic acid is used, e.g., to deliver a therapeutic gene or siRNA to a selected tissue, e.g., the pancreas. For example, local delivery, e.g., by infusion to the selected tissue, can be used. In addition, cells, preferably autologous cells, can be engineered to express a selected gene sequence (e.g., HRD1 or a functional fragment thereof), and can then be introduced into a subject in positions appropriate for the amelioration of the symptoms of an ER stress-related disorder, e.g., islet cells inserted into the pancreas to treat diabetes. Alternately, cells from a MHC matched individual can be utilized. The expression of the selected gene sequences is typically controlled by appropriate gene regulatory sequences to allow expression in the necessary cell types. Such gene regulatory sequences are well known to the skilled artisan. Such cell-based gene expression techniques are well known to those skilled in the art, see, e.g., Anderson, U.S. Pat. No. 5,399,349.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

Dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population), and confirmed in clinical trials. The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

The pharmaceutical compositions can be included in a container, pack, or dispenser together with instructions for administration.

This invention is further illustrated by the following examples which should not be construed as limiting. The contents of all references, patents and published patent applications cited throughout this application are incorporated herein by reference.

### EXAMPLES

### Example 1

### XBP-1 Splicing Assay

RNA from cells was reverse transcribed using Oligo-dT primer. PCR is performed using primers shown in Table 2.

TABLE 2
RT-PCT primers

| | | | SEQ |
|---|---|---|---|
| | Sense (S) or | | ID |
| Species | Anitsense (AS) | Sequence | NO: |
| Human | hXBP-1.1S | AAACAGAGTAGCAGCTCAGACTGC | 8 |
| Human | hXBP-1.2AS | TGGGCAGTGGCTGGATGAAAGC | 9 |
| Mouse | mXBP-1.3S | AAACAGAGTAGCAGCGCAGACTGC | 10 |
| Mouse | mXBP-1.6AS | CAGACAATGGCTGGATGAAAGC | 11 |
| Rat | rXBP-1.3S | AAACAGAGTAGCAGCACAGACTGC | 12 |
| Rat | mXBP-1.6AS | CAGACAATGGCTGGATGAAAGC | 11 |

These primers amplify a 768-base pair PCR product for human, a 774-base pair PCR product for mouse, and a 774-base pair PCR product for rat from the unspliced XBP-1, and 742-base pair (human) and 748-base pair (mouse, rat) PCR products from the spliced form. These primers were designed to amplify the region encompassing the splice junction of XBP-1 mRNA.

Reverse Transcriptase-PCR(RT-PCR) was performed using mRNA isolated using standard methods from a wild-type mouse fibroblast cell line and Ire1α:Ire1β double knock-out cell line. The cells were treated with tunicamycin or thapsigargin for 4 or 8 hours. Tunicamycin causes ER stress experimentally by blocking N-linked glycosylation, which is a crucial step for protein folding in the ER. Thapsigargin also induces ER stress experimentally by altering calcium ion concentrations in the ER.

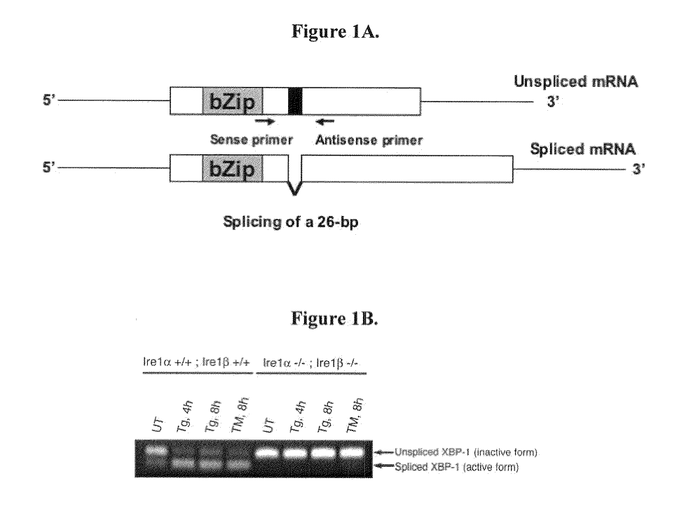

The results are illustrated in FIG. 1B. The 26 base pair size difference between the two forms, spliced and unspliced, was visualized by running the PCR product on 2.5% agarose gel (FIG. 1B). The thermal cycle reaction was performed as follows: 94° C. for 3 minutes, followed by 35 cycles at 94° C. for 1 minute, 62° C. for 1 minute, 72° C. for 1 minute, and 72° C. for 10 minutes. RT-PCR analysis detected predominantly smaller fragment corresponding to active form (spliced form) of XBP-1 mRNA in wild-type cell line treated with tunicamycin or thapsigargin. In contrast, the same analysis detected only inactive form of XBP-1 mRNA in Ire1α−/−:Ire1β−/− double knock-out cell line (FIG. 1B).

### Example 2

### XBP-1 Splicing Assay with Pst I Digestion

A Pst I restriction site is removed by IRE1-mediated cleavage and splicing of the mRNA, thus, the results of the experiment described in Example 1 can also be achieved using an intermediate step of Pst I cleavage to facilitate distinguishing between spliced and unspliced XBP-1. Pst I digestion of the spliced form of XBP-1 yields a 768-base pair fragment for human, 774-base pair fragment for mouse and rat. The unspliced forms of XBP-1 yield 285 base pair and 483 base pair fragments for human, 291 base pair and 483 base pair fragments for mouse and rat.

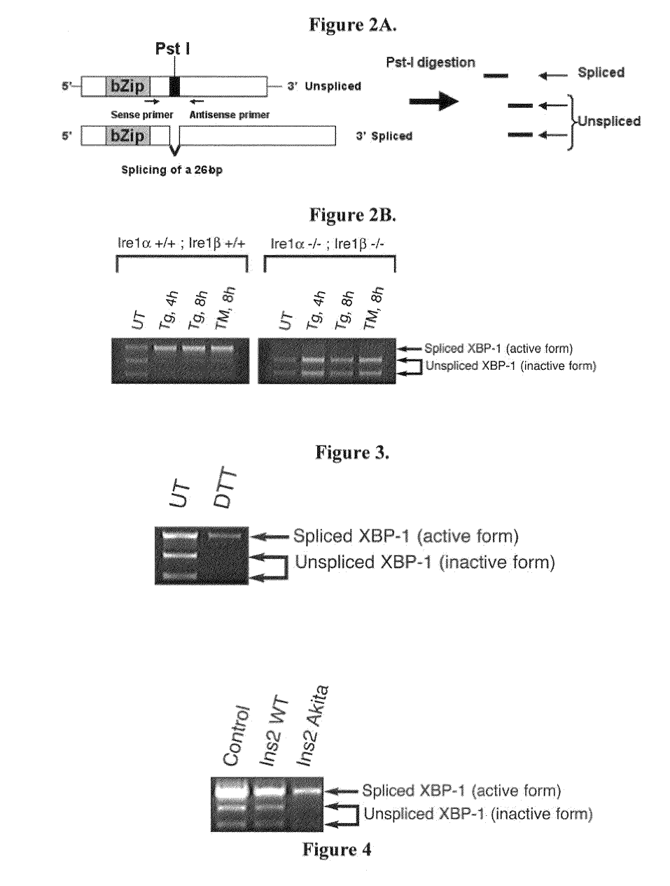

RT-PCR performed as described in Example 1 was followed by Pst I digestion, and the digested products were visualized on a 2% agarose gel. Since the intron removed by IRE1-mediated splicing contains the Pst I site, the spliced form (the active form) of XBP-1 mRNA (cDNA) loses its Pst I site after IRE1 processing. Pst I digestion of RT-PCR product produces undigested larger fragment corresponding to the active form (spliced form, no Pst I site) of XBP-1 mRNA and two smaller, digested fragments corresponding to the inactive form (unspliced form, which retains the Pst I site) (FIG. 2A). Pst I digestion of RT-PCR product generated as described above detected predominant non-digested fragment corresponding to active form (spliced form) of XBP-1 mRNA in wild-type cell line treated with tunicamycin or thapsigargin. In contrast, the same analysis detected only inactive form of XBP-1 mRNA in Ire1α−/−:Ire1β(3−/− double knock-out cell line (FIG. 2B).

### Example 3

### ER Stress Signaling is Activated in Islet Cells under Physiological Conditions

To determine whether ER stress signaling is activated in islet cells under physiological conditions, XBP-1 splicing was monitored in freshly isolated mouse islet cells, using the methods described above in Example 2. The results are shown in FIG. 3. High levels of XBP-1 mRNA splicing were detected in the islet cells. Dithiothreitol (DTT) treatment enhanced the XBP-1 splicing. It is known that DTT blocks disulfide bond formation experimentally, resulting in ER stress. These results illustrate that XBP-1 splicing, and hence ER stress, occurs in islet cells under physiological conditions. This demonstrates that the methods described herein can be successfully used to detect and measure ER stress under physiological conditions; in addition, as the islet cells secrete insulin, this demonstrates that ER stress may play a role in the etiology of diabetes.

### Example 4

### XBP-1 Splicing Assay Using Quantitative Real-Time PCR

This example describes a method to quantify the expression levels of spliced form and unspliced form of XBP-1 mRNA using real-time PCR. Briefly, RNA from cells was reverse transcribed using Oligo-dT primer. PCR was performed using primers shown in Table 3.

TABLE 3
Real-Time PCR primers

| Species | | | SEQ |
|---|---|---|---|
| of Target | Sequence Seq. Name | ID NO: | |
| Human | CAGCACTCAGACTACGTGCA | hXBP1.3S: | 13 |
| | ATCCATGGGGAGATGTTCTGG | hXBP1.6AS: | 14 |
| | CTGAGTCCGAATCAGGTGCAG | mXBP1.11S: | 15 |
| Mouse | CAGCACTCAGACTATGTGCA | mXBP1.7S | 16 |
| | GTCCATGGGAAGATGTTCTGG | mXBP1.10AS | 17 |
| | CTGAGTCCGAATCAGGTGCAG | mXBP1.11S | 15 |
| Rat | ATCCATGGGAAGATGTTCTGG | rXBP1.6AS | 18 |
| | CAGCACTCAGACTACGTGCG | rXBP1.7S | 19 |
| | CTGAGTCCGAATCAGGTGCAG | mXBP1.11S | 15 |

To amplify the active form of XBP-1 mRNA, mXBP1.11S and hXBP1.6AS (human target), mXBP1.11S and mXBP1.10AS (mouse target) and mXBP1.11 S and rXBP1.6AS (rat target) were used. Two mismatches to the native XBP-1 sequence were introduced in the mXBP1.11S primer to reduce background signal.

To amplify the inactive form of XBP-1, hXBP-1.3S and hXBP1 (human target), mXBP1.7S and mXBP1.10AS (mouse target), and rXBP1.7S and rXBP1.6AS (rat target) were used.

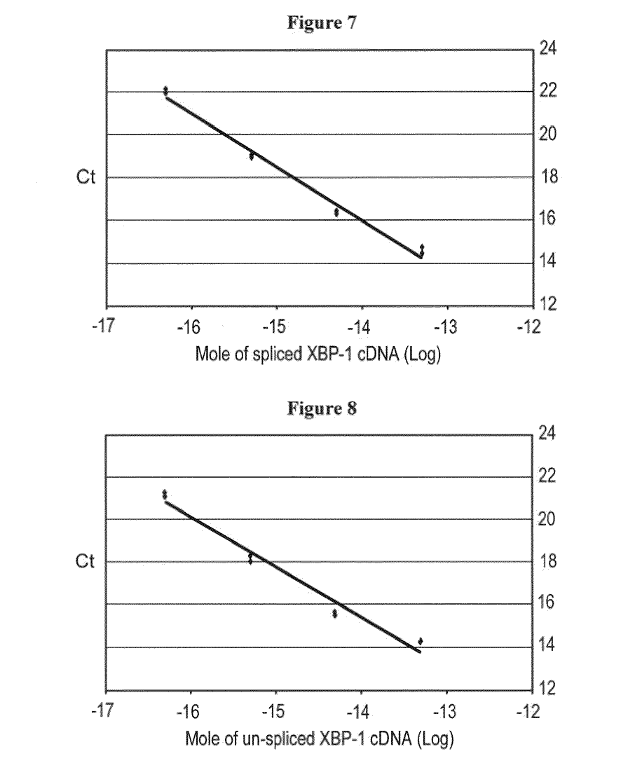

The results using mouse XBP-1 cDNA as a target are illustrated in FIGS. 7 and 8. The thermal cycle reaction was performed using ABI prism 7000 sequencer detection system as follows: 50° C. for 2 minutes, 95° C. for 10 minutes, followed by 40 cycles at 95° C. for 15 seconds and 60° C. for 1 minute. Standard curves for the amplification of the XBP-1 target detected using a cybergreen-labeled probe are shown in FIGS. 7 and 8. Ct is the threshold cycle. The threshold cycle is when the system begins to detect the increase in the signal associated with an exponential growth of PCR product during the log-linear phase.

### Example 5

### XBP-1 Splicing Assay Using XBP-1-GFP Fusion Protein

XBP-1 splicing has also been detected using an XBP-1-GFP fusion protein. Briefly, human XBP-1 partial cDNA (without the stop codon) was cloned into pEGFP-N1 (CLONTECH). Under ER stress conditions, the EGFP was expressed as a fusion to the C-terminus of spliced XBP-1, because the spliced form is in the same reading frame as EGFP and there are no intervening stop codons. Under normal conditions, i.e., non-ER stress conditions, the EGFP is not expressed, as the EGFP is not in frame with the unspliced form of XBP-1.

### Example 6

### Anti-Phospho IRE1α Antibodies

To directly quantify IRE1 activity levels, antibodies against the phosphorylated and non-phosphorylated forms of IRE1α were generated. Peptide sequences used as immunogens to generate the antibodies are listed in Table 4. The phosphorylation site of Ire1α is conserved from lower eukaryotes to humans (Shamu and Walter, Embo J 15:3028-39 (1996); Tirasophon et al., Genes Dev 12:1812-24 (1998)).

TABLE 3
Peptide Sequences for Generating
anti-Phospho IRE1α antibody

| Peptide Sequence | SEQ ID NO: | Antigen |
|---|---|---|
| CVGRH[pS]FSRRSG | 20 | Phospho IRE1α |
| CVGRHSFSRRSG | 21 | IRE1α |

The antibodies were produced using standard methodology. Briefly, the indicated phosphopeptides were synthesized, multi-link conjugated to KLH, and individually immunized following a 90-day protocol, using two specific pathogen free (SPF) rabbits. Four immunizations were performed per rabbit, with varying dosage. The antibody was prepared from bulk antiserum by affinity purification followed by adsorption against the non-phospho analog column peptide.

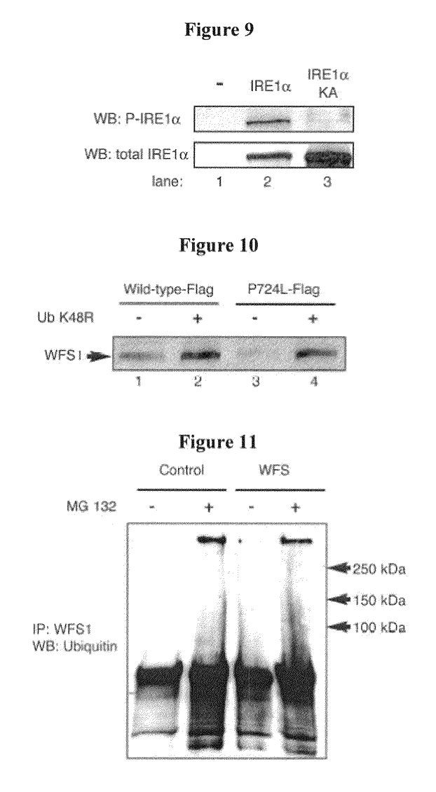

The specificity of the antibody PIRE1A1 was tested by immunoblot analysis of wild-type or kinase inactive K599A human IRE1α expressed in COS7 cells. PIRE1A1 antibody specifically detects wild-type IRE1α which is known to be autophosphorylated by over-expression (Urano et al., Science 287:664-6 (2000)). PIRE1A1 antibody specifically detects the phosphorylated form of IRE1α protein. Immunoblot analysis of wild-type and kinase inactive K599A (IRE1aKA) human IRE1α expressed in COS7 cells using PIRE1A1 antibody (P-IRE1α) or total IRE1α antibody. As shown in FIG. 9, PIRE1A1 antibody specifically detects wild-type IRE1α which is known to be autophosphorylated by over-expression. The amount of total IRE1α is shown in the lower panel.

Immunoblot analysis of phosphorylated IRE1α using lysates from different cell lines showed highest expression of the protein in the pancreatic β-cell cell line, MIN6. Using the PIRE1A1 antibody, the ER stress level in a mouse insulinoma cell line MIN6 expressing the pathogenic P724L and G695 WFS1 mutants was examined. IRE1a phosphorylation level was higher in cells expressing WFS1 mutants than in cells expressing wild-type WFS1, indicating that expression of the pathogenic WFS1 mutants causes ER stress and activates IRE1 signaling. In addition, the viability of MIN6 cells expressing mutant forms of WFS1 was lower than that of cells expressing wild-type WFS1. This suggests that expression of mutant forms of WFS1 is toxic to β cells.

### Example 7

### Effect of the P724L Mutation in the Wolfram Gene WFS1 on Cellular Localization

The experiments in this Example evaluated the effect of the P724L mutation of WFS1 on cellular localization of wild-type and mutant WFS1.

Plasmids, Cell Culture, and Transfection

Full-length human WFS1 cDNA and P724L mutant WFS1 cDNA was tagged with a Flag epitope and subcloned each to a pcDNA3 plasmid under the control of the cytomegalovirus promoter using standard molecular biology methods. The P724L mutation was introduced using the GeneTailor Site-Directed Mutagenesis System (Invitrogen, Carlsbad, Calif.). COS7 cells were transfected using FuGene (Roche, Basel) and maintained in DMEM with 10% fetal bovine serum.

Immunostaining

Cells were fixed in 2% paraformaldehyde for 30 min at room temperature, then permeabilized with 0.1% Triton X-100 for 2 minutes. The fixed cells were washed with PBS, blocked with 10% BSA for 30 min, and incubated in primary antibody overnight at 4° C. The cells were washed 3 times in PBS/Tween™ 0.1% and incubated with secondary antibody for 1 hour at room temperature. Images were obtained with a Leica TCS SP2 AOBS Confocal Microscope with LCS Software.

Results:

The cellular localization of wild-type and mutant WFS1 was determined by immunostaining cells transfected with an expression vector for wild-type or P724L WFS1 tagged at its C-terminus with a Flag epitope. Immunostaining of cells expressing wild-type WFS1 with anti-Flag antibody showed a diffuse reticular pattern that co-localized with the ER marker ribophorin I. However, immunostaining of cells expressing mutant WFS1 with anti-Flag antibody showed a punctate staining pattern in the ER, suggesting that WFS1 tends to aggregate there. Part of WFS1^{P724L} showed a diffuse reticular pattern and was co-localized with ribophorin I, suggesting that this part of WFS1^{P724L} is localized to the ER membrane. However, the signal intensity of mutant WFS1 was much lower than that of wild-type WFS1. These staining patterns suggest that in contrast to wild-type WFS1, most of the newly synthesized WFS1^{P724L} protein aggregates and thus is not expressed on the ER membrane. This accumulation results in ER stress, and is likely to be analogous to the etiology of Wolfram syndrome.

### Example 8

### Effect of the P724L Mutation in the Wolfram Gene WFS1 on Expression Levels, Ubiquitination, and Aggregation

The experiments described in this Example evaluated the effect of the P724L mutation of WFS1 on expression levels, ubiquitination, and aggregation of mutant WFS1.

Immunoblotting

The cells described in Example 7 were lysed in ice-cold buffer (20 mM Hepes, pH 7.5, 1% Triton X-100, 150 mM NaCl, 10% glycerol, 1 mM EDTA) containing protease inhibitors for 15 minutes on ice. Insoluble material was recovered by centrifugation at 13,000 g for 15 minutes and solubilized in 10 mM Tris-HCl and 1% SDS for 10 min at room temperature. After the addition of 4 volumes of lysis buffer, samples were sonicated for 10 seconds. Lysates normalized for total protein (20 mg per lane) were separated using 4%-20% linear gradient SDS-PAGE (Bio Rad, Hercules, Calif.) and electroblotted.

Results:

Measuring the steady-state expression level of WFS1P724L by immunoblot analysis, we found that it did not accumulate to high levels in transfected cells, suggesting that WFS1P724L was subject to increased intracellular degradation.

The WFS mutant was then co-expressed with a dominant negative form of ubiquitin to determine whether or not polyubiquitination is required for WFS1P724L degradation. The Lys-48 residue of ubiquitin, which is the site of isopeptide linkage of other ubiquitin molecules, is essential for the formation of multi-ubiquitin chains. Mutant ubiquitin in which this invariant lysine is replaced by the arginine (K48R) is a polyubiquitin chain terminator that reduces the efficiency of proteasome-mediated degradation and stabilizes polyubiquitinated substrates (Chau et al., Science 243, 1576-1583 (1989); Finley et al., Mol Cell Biol 14, 5501-5509 (1994)). Co-expression of WFS1P724L and ubiquitin^{K48R} increased the WFS1P724L expression level as well as the wild-type WFS1 expression level (FIG. 10), suggesting both are degraded by the ubiquitin-proteasome system.

To analyze the ubiquitination level of mutant WFS1 protein in Wolfram syndrome, detergent-soluble lysates were immunoprecipitated from the fibroblasts of a patient with this syndrome, using a polyclonal antibody to WFS1, then immunoblotted with a monoclonal antibody to ubiquitin. The patient was a compound heterozygote for G695V and W648X. The W648X mutation predicts premature termination and a lack of 242aa of the C-terminus of WFS1 protein. Ubiquitin reactivity was increased in proteasome inhibitor MG132-treated cells and was higher in the patient's cells than in control cells (FIG. 11), indicating that mutant WFS1 protein is more susceptible to ubiquitination than wild-type WFS1 protein.

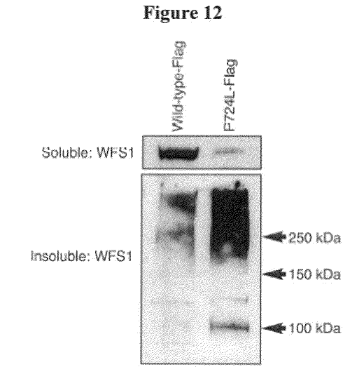

The aggregation of WFS1P724L was assessed by SDS-PAGE immunoblot analysis of detergent-soluble and detergent-insoluble lysates from COS7 cells transiently expressing these proteins. The formation of insoluble and high-molecular-weight complexes was much more prominent in cells expressing WFS than in cells expressing wild-type WFS1 (FIG. 12, lower panel). This suggests that mutant WFS1 tends to mis-fold and form insoluble aggregates in the ER.

These results suggest that mutant WFS1 proteins in patients with Wolfram syndrome are degraded by the ubiquitin-proteasome pathway, but some of them form insoluble aggregates that accumulate in the ER. This accumulation results in ER stress, which is likely to cause the β cell death associated with Wolfram syndrome.

### Example 9

### Effect of the P724L Mutation in the Wolfram Gene WFS1 on Degradation

The experiments described in this Example evaluated the effect of the P724L mutation of WFS1 on degradation of mutant WFS1. EDEM is a type II ER transmembrane protein having homology to class I a1,2-mannosidase, which is involved in N-glycan processing (Hosokawa et al., EMBO Rep 2, 415-422 (2001)). It has been shown that EDEM is directly involved in the ERAD system for glycoproteins (Hosokawa et al., 2001, supra; Hosokawa et al. J Biol Chem 278(28):26287-94 (2003); Molinari et al., Science 299, 1397-1400 (2003); Oda et al., Science 299, 1394-1397 (2003)). Because WFS1 is a glycoprotein localized to the ER, the involvement of EDEM in the degradation of WFS1P724L was evaluated.

Results:

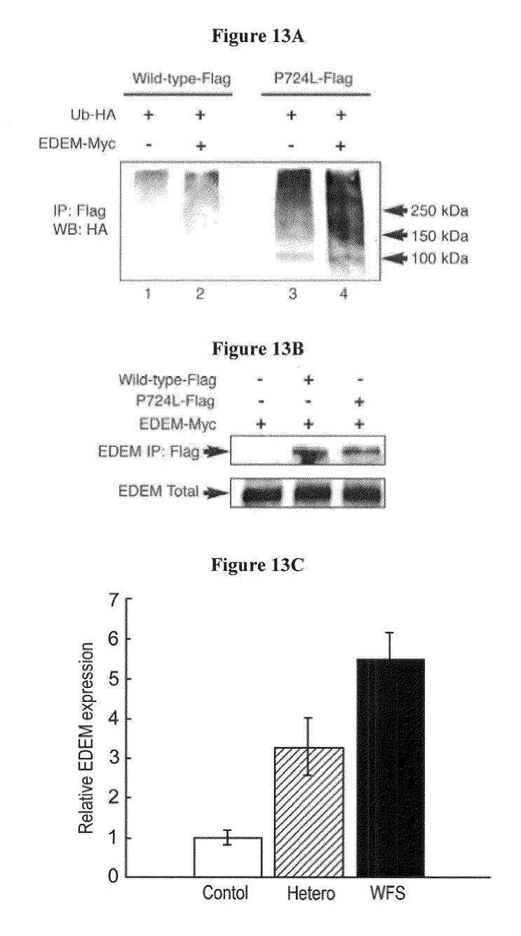

To determine whether WFS1 is ubiquitinated by EDEM, Myc-tagged EDEM and either wild-type or P724L WFS1 was co-transfected with HA-tagged ubiquitin in COS7 cells. EDEM expression increased the ubiquitination of both wild-type and P724L WFS1. However, a higher level of ubiquitination occurred in cells expressing WFS1P724L than in cells expressing wild-type WFS1 (FIG. 13A). To test the association between WFS1 and EDEM, Myc-tagged EDEM and Flag-tagged WFS1P724L were co-transfected into COST cells, and these cells were subjected to co-immunoprecipitation analysis. Both wild-type and P724L WFS1 were associated with EDEM (FIG. 13B), suggesting that EDEM is involved in the degradation of WFS1 proteins. These results indicate that both wild-type and mutant WFS1 are degraded by the ERAD system, but that the mutant WFS1 is more susceptible to degradation by the EDEM-ERAD pathway.

To measure the activity level of the ERAD system in patients with Wolfram syndrome, quantitative real-time PCR was used to compare EDEM expression in lymphoblasts from patients and their relatives who were homozygous or heterozygous normal for the WFS1 mutation. As compared to patients' relatives who were homozygous normal, patients who were homozygous for the WFS1 mutation had 6 to 7 times higher average levels of EDEM messenger RNA, while patients' relatives who were heterozygous for this mutation had levels that were 4 to 5 times higher (FIG. 13C).

These findings indicate that the ERAD system is highly activated in patients with Wolfram syndrome.

### Example 10

### Effect of the P724L Mutation in the Wolfram Gene WFS1 on ER Stress

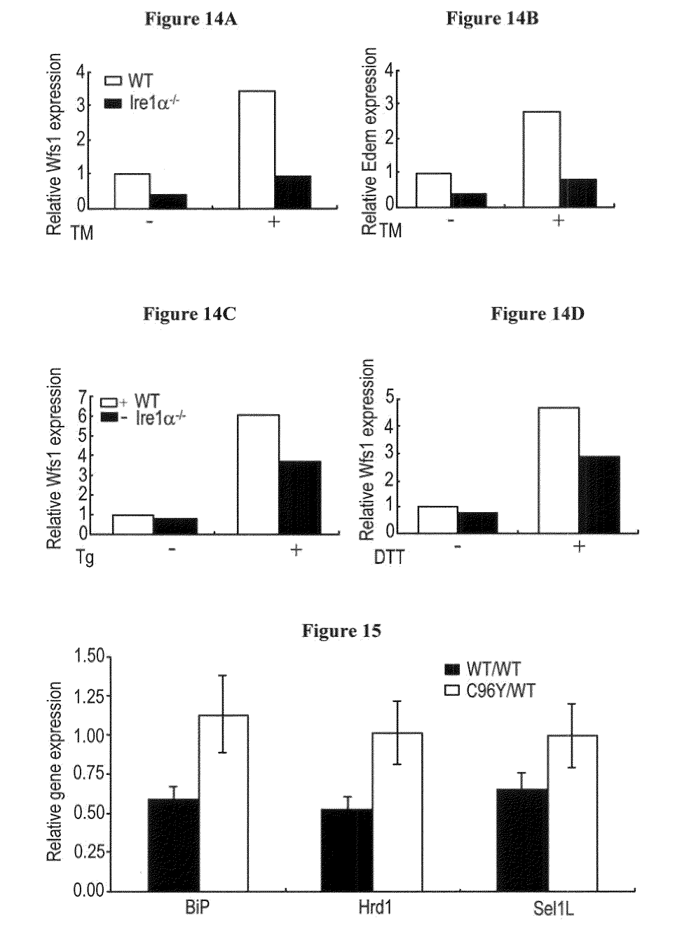

As noted above, WFS1 encodes an ER-resident transmembrane protein. Membrane proteins in the ER are often involved in the unfolded protein response (UPR), a system that mitigates intracellular stress caused by the accumulation of misfolded proteins in the ER (Harding et al., Annu. Rev. Cell. Dev. Biol. 18:575-99 (2002); Patil and Walter, Curr. Opin. Cell. Biol. 13:349-55 (2001)). By measuring the expression level of WFS1 under ER stress, it has been found that WFS1 mRNA is induced by this stress and is under control of inositol requiring 1 (IRE1), a central component of the UPR (FIG. 14A-D). This suggests that WFS1 is also a component of the UPR and may be protective against ER stress.

Real-Time Polymerase Chain Reaction

Total RNA was isolated from the cells described in Example 7 by the guanidine-thiocyanate-acid-phenol extraction method, reverse transcribing 1 mg of total RNA from cells with Oligo-dT primer. For the thermal cycle reaction, the ABI prism 7000 sequencer detection system (Applied Biosystems, Foster City, Calif.) was used at 50° C. for 2 min, 95° C. for 10 min, then 40 cycles at 95° C. for 15 sec and at 60° C. for 1 min. The polymerase chain reaction (PCR) in triplicate for each sample and all experiments were repeated twice, using human GAPDH as a control. The following set of primers and Cyber Green (Applied Biosystems) for real-time PCR: for human endoplasmic reticulum degradation-enhancing alpha-mannosidase-like protein (EDEM), CAAGTGTGGGTACGCCACG (SEQ ID NO:22) and AAAGAAGCTCTCCATCCGGTC (SEQ ID NO:23); for mouse EDEM, CTACCTGCGAAGAGGCCG (SEQ ID NO:24) and GTTCATGAG CTGCCCACTGA (SEQ ID NO:25); and for mouse WFS1, CCATCAACATGCTCCCGTTC (SEQ ID NO:26) and GGGTAGGCCTCGCCATACA (SEQ ID NO:27).

Results:

Quantitative real-time PCR of WFS1 using reverse-transcribed RNA from wild-type (WT) and Ire1α knock-out (Ire1α−/−) mouse embryonic fibroblast cells. Cells were untreated or treated with tunicamycin (TM) (FIG. 14A-B), thapsigargin (TG) (FIG. 14C) or dithiothreitol (DTT) (FIG. 14D) for six hours. EDEM expression in TM-treated cells was also shown as control (FIG. 14B). The amount of mouse WFS1 and EDEM mRNA was normalized to the amount of GAPDH mRNA in each sample.

The results described herein indicate that mutant WFS1 protein in patients with Wolfram syndrome forms insoluble high-molecular complexes that may be toxic to the cells. These findings suggest that the pathogenesis of Wolfram syndrome can be attributed to the combined effects of the lack of functional WFS1 protein and the presence of aggregated WFS1 proteins in cells.

### Example 11

### Insulin-2 Mutation in the Akita Mouse Causes ER Stress

Pancreatic β-cell death contributes to both type 1 and type 2 diabetes. Recent observations suggest that chronic ER stress in β cells plays a role in the pathogenesis of diabetes (Harding and Ron, Diabetes 51(Suppl. 3):5455-461 (2002)). Moreover, recent reports suggest that ER stress has an important role in β-cell death in the Akita mouse model for diabetes (Kayo and Koizumi, J. Clin. Invest. 101:2112-2118 (1998); Yoshioka et al., Diabetes 46:887-894 (1997); Oyadomari et al., J. Clin. Invest. 109:525-532 (2002)). The Akita mouse is a C57BL/6 mouse that is heterozygous for a mutation in the insulin 2 gene that results in an amino acid substitution, cysteine 96 to tyrosine (Ins2^{WT/C96Y}) (Wang et al., J. Clin. Invest. 103:27-37 (1999)). Cysteine 96 is involved in the formation of one of the two disulfide bonds between the A and B chains of mature insulin (Masharani and Karam, in Greenspan, F. S., and Gardner, D. G., (Eds.), McGraw-Hill, 2001, pp. 623-698.). It is likely that this mutation causes misfolding of the insulin precursor in the ER of (3 cells. Therefore, it is important to quantify ER stress levels in the β cells of Akita mice to monitor their disease status. In this study, we measured the expression levels of ER stress markers and components of the ERAD system in the islets of Akita mice by quantitative real-time polymerase chain reaction (PCR).

Diabetes in the Akita mouse is accompanied by neither obesity nor insulitis. These mice spontaneously develop diabetes with dramatic reduction in beta-cell mass. Symptoms include hyperglycemia, hypoinsulinemia, polydipsia, and polyuria, beginning around 4 weeks of age. This condition in the Akita mouse is termed diabetes.

A. BiP, Hrd1, and Sel1L Levels in Pancreatic Islet Cells from Akita Mice

Diabetes in the Akita mouse is not associated with obesity or insulitis; rather, it develops spontaneously with dramatic reduction in β-cell mass (Kayo and Koizumi, J. Clin. Invest. 101:2112-2118 (1998); M. Yoshioka et al., Diabetes 46:887-894 (1997)). Recent observations support the idea that ER stress causes β-cell death and thus leads to diabetes in the Akita mouse (Ins2^{WT/C96Y}) (Oyadomari et al., J. Clin. Invest. 109:525-532 (2002); Urano et al., Science 287:664-666 (2000); Nishitoh et al., Genes Dev. 16:1345-1355 (2002)). It has been shown that the ER-resident molecular chaperone BiP (Binding Protein) is upregulated in the pancreas of the Akita mouse (Oyadomari et al., 2002, supra).

Isolating Islet Cells from Mouse Pancreas

Islet cells were handpicked from collagenase P-digested whole pancreas according to a standard method (Lacy and Kostianovsky, Diabetes 16:35-39 (1967)). Briefly, after the mice were anesthetized by intraperitoneal injection of sodium pentobarbital, pancreatic islets were isolated by pancreatic duct injection of 500 U/ml of collagenase solution, then digested at 37° C. for 40 minutes with mild shaking. Islet cells were washed several times with HBSS, separated from acinar cells on a discontinuous Ficoll 400 gradient, and then selected by eye under a dissecting microscope. Freshly isolated islets were cultured for 14 hours in RPMI 10% FCS (Andersson, Diabetologia 14:397-404 (1978)).

Immunoblotting and Immunoprecipitation

Islet cells were lysed in ice-cold buffer (20 mM Hepes, pH 7.5, 1% Triton X-100, 150 mM NaCl, 10% glycerol, 1 mM EDTA) containing protease inhibitors for 15 min on ice, then clarified them by centrifugation at 14,000 g for 10 min. Lysates were normalized for total protein, 20 mg per lane, separated using 4%-20% linear gradient SDS-PAGE, then electroblotted to nitrocellulose membranes. The anti-HRD1 antibody was raised in rabbits immunized with a KLH-conjugated synthetic peptide, TCRMDVLRASLPAQS (SEQ ID NO:28). Flag M2 antibody and HA antibody were purchased respectively, from Sigma (St. Louis, Mo.) and Roche. The lysates were immunoprecipitated with the indicated antibodies and separated using 4%-20% linear gradient SDS-PAGE (Bio Rad, Hercules, Calif.).

Results:

Hrd1 (Hydroxymethylglutaryl Reductase Degradation 1) and Sel1L (Suppressor/Enhancer of Lin-12) are components of the ERAD system. In this study, it was found that BiP, Hrd1, and Sel1L were all upregulated in pancreatic islet cells from Akita mice (FIG. 14), strongly suggesting that these cells are under ER stress.

B. XBP-1 Splicing Levels Measured in Mouse Insulinoma Cells

Since the phenotype of the Akita mouse is caused by a mutation which can cause conformational changes in the insulin 2 (Ins2) gene product (Wang et al., J., 1999. J. Clin. Invest. 103:27-37), it is hypothesized that pancreatic cells in Akita mice are under ER stress, and this stress can cause beta cell death. To initially test this hypothesis, XBP-1 splicing levels were measured in mouse insulinoma cells (MIN6 cells) expressing either an Ins2 gene with the Akita mutation or a wild-type insulin-2 gene. The MIN6 cells were cultured in 10 cm collagen-coated dishes in DMEM supplemented with 25 mM glucose and 15% FCS. Plasmids encoding the wild-type or mutant Ins-2 genes were transfected into the cells using FUGENE™ transfection reagent following the manufacturer's instructions (Roche, Basel, Switzerland).

Real-time Polymerase Chain Reaction

To isolate total RNA from the cells, the guanidine thiocyanate-acid-phenol extraction method was used, in which 1 mg of total RNA from cells is reverse transcribed using Oligo-dT primer. During PCR, XBP-1 mRNA was used. Primers were mXBP1.11S: CTGAGTCCG AATCAGGTGCAG (SEQ ID NO:15), and mXBP1.10AS: GTCCATGGGAAGATGTTCTGG (SEQ ID NO:17). To reduce the background signal, two mismatches were introduced to the native XBP-1 sequence in mXBP1.11S. To amplify the spliced form of mouse XBP-1, mXBP1.7S: CAGCACTCAGACTATGTGCA (SEQ ID NO:16) and mXBP1.10AS were used. In amplification procedures, mBiP.3S: TTCAGCCAATTATCAGCAAACTCT (SEQ ID NO:29) and mBiP.4AS: TTTTCTGATGTATCCTCTTCACCAGT (SEQ ID NO:30) primers were used for mouse BiP, mHRD1.1S: CCTGCTTGTGAGTATGGGACC (SEQ ID NO:31) and mHRD1.2AS: TGGGTTTCCACAGTTGGGAA (SEQ ID NO:32) primers were used for Hrd1, and mSEL1.1S: ACAGCCTTAACCAACTTGAGGTG (SEQ ID NO:33) and mSEL1.2AS: TCCGGGAAGCAACGAATCTA (SEQ ID NO:34) primers were used for Sel1L. For the thermal cycle reaction, the ABI prism 7000 sequencer detection system was used to incubate the samples at 50° C. for 2 minutes, and then 95° C. for 10 minutes, followed by 40 cycles at 95° C. for 15 seconds and 60° C. for 1 minute.

Results:

The results are shown in FIG. 4. High XBP-1 splicing levels, which reflected high ER stress levels, were detected in the MIN6 cells expressing mutant insulin 2 gene. These results indicate that the methods described herein can be used to detect differences in ER stress levels correlating with disease states.

C. IRE1 Activity Levels in Islets of Akita Mice

It has been shown that the upregulation of the ERAD components is regulated by the IRE1-XBP-1 pathway. To further examine the involvement of IRE1 signaling in upregulation of ERAD genes, the IRE1 activity level in the islets of Akita mouse was measured. XBP-1 mRNA splicing level, which reflects the IRE1 activity level, was used to quantify the IRE1 activity level, as described herein.

To test this method, the ratio between spliced and unspliced XBP-1 expression levels was measured in mouse embryonic fibroblasts treated for 2 hours with tunicamycin, an ER stress inducer. The ratio of spliced XBP-1 mRNA expression to unspliced XBP-1 mRNA expression was measured.

Results:

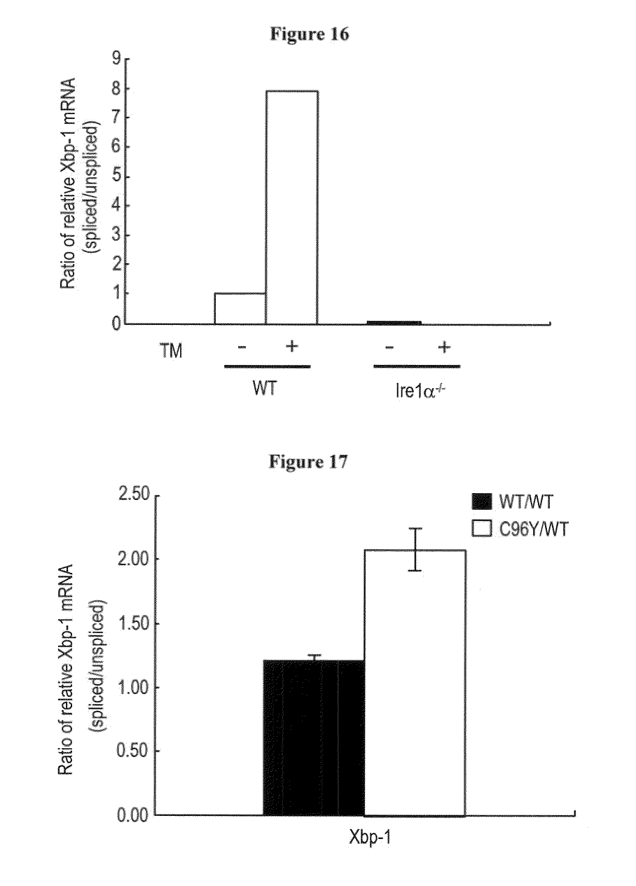

The induction of XBP-1 splicing by ER stress was measurable in wild-type, but not in Ire1α knock-out (Ire1α−/−) mouse embryonic fibroblasts (FIG. 16). Because there is no XBP-1 splicing in Ire1a knock-out (Ire1α−/−) mouse embryonic fibroblasts (Calfon et al., Nature 415:92-96 (2002)), this result further validates the methods described herein. The XBP-1 splicing levels were higher in Akita mice than in control animals (FIG. 17). The data also support the prediction that the ER stress level is higher in the islets of Akita mice than in those of control mice.

D. Stability of Mutant Insulin in Akita Mice

Upregulation of the ERAD components Hrd1 and Sel1L prompted the examination of the stability of mutant insulin in Akita mice.

Briefly, COS7 cells were transfected with wild-type and mutant insulin-2 expression vectors, then the steady-state expression level of mutant insulin, Ins2^{C96Y}, was measured by immunoblot analysis as described herein, in untreated cells and in cells treated with the proteasome inhibitor MG132. In addition, Ins2^{C96Y} was co-expressed with a dominant negative form of ubiquitin to determine whether or not polyubiquitination is required for Ins2^{C96Y} degradation. The Lys-48 residue of ubiquitin, which is the site of isopeptide linkage of other ubiquitin molecules, is essential for the formation of multi-ubiquitin chains. Mutant ubiquitin in which this invariant lysine is replaced by arginine (K48R, referred to as ubiquitin^{K48R}) is a polyubiquitin chain terminator that reduces the efficiency of proteasome-mediated degradation and stabilizes polyubiquitinated substrates (Finley et al., Mol. Cell. Biol. 14:5501-5509 (1994)).

Results:

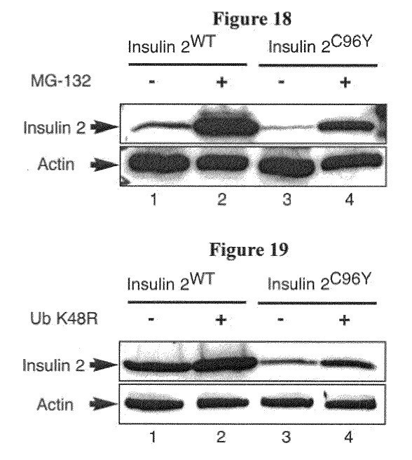

As shown in FIG. 18, Ins2^{C96Y} does not accumulate to high levels in transfected cells, suggesting that it was subject to increased intracellular degradation. The expression level of mutant insulin was increased in cells treated with MG132, suggesting that the ubiquitin-proteasome pathway is involved in the degradation of mutant insulin (FIG. 18). Co-expression of Ins2^{C96Y} and ubiquitin^{K48R} increased the Ins2^{C96Y} expression level (FIG. 19), indicating that Ins2^{C96Y} is degraded by the ubiquitin-proteasome system. Thus, accumulation of large amounts of insulin is likely to lead to ER stress.

E. Ins2^{C96Y} Mutant Insulin-2 is Susceptible to HRD1-Mediated Ubiquitination and Degradation

Because HRD1 is upregulated in the islets of Akita mice and encodes an E3 ubiquitin ligase required for the ERAD system (Kaneko et al., FEBS Lett. 532:147-152 (2002); Kikkert et al., J. Biol. Chem. 279:3525-3534 (2004); Nadav et al., Biochem. Biophys. Res. Commun. 303:91-97 (2003); Bays et al., Nat. Cell Biol. 3:24-29 (2001)), the question of whether or not mutant insulin is ubiquitinated by HRD1 was explored.

Plasmids, Cell Culture, and Transfection

The plasmid HRD1-pCMVSPORT6 was obtained from Open Biosystems (Huntsville, Ala.). K. Tanaka provided ubiquitin-Flag-pcDNA3; H. Nishitoh provided insulin-2-HA-pcDNA3 and insulin-2 C96Y-HA-pcDNA3, while S. Oyadomari provided insulin-2-pcDNA and insulin-2 C96Y-pcDNA. COS7 cells and HeLa cells were maintained in DMEM with 10% fetal bovine serum and transfected using FUGENE™ (Roche, Basel) and HELA MONSTER™ transfection reagents (Minis, Madison, Mich.), respectively. The COS7 cells were co-transfected with HRD1 expression vector and either wild-type or C96Y insulin-2 with Flag-tagged ubiquitin.

Flag M2 antibody and HA antibody were purchased respectively, from Sigma (St. Louis, Mo.) and Roche. The lysates were immunoprecipitated with the HA antibody and separated using 4%-20% linear gradient SDS-PAGE (Bio Rad, Hercules, Calif.). Western blotting using an anti-FLAG antibody was used to detect ubiquitination levels.

Results:

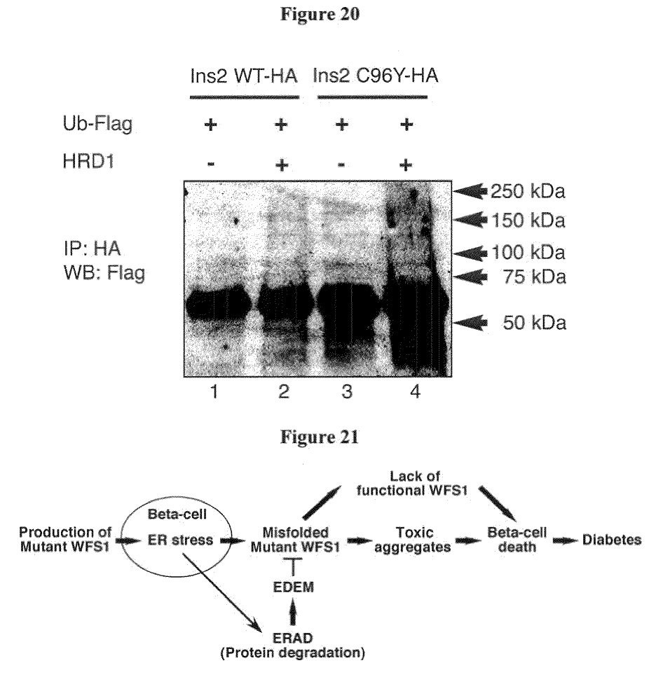

HRD1 expression did not increase the ubiquitination of wild-type insulin-2, but did increase that of C96Y insulin-2 (FIG. 20), demonstrating that mutant insulin-2 is susceptible to HRD1-mediated ubiquitination and degradation.

### SUMMARY

Taken together, these findings suggest that misfolded insulin produced in Akita mice is selectively ubiquitinated and degraded by an HRD1-mediated ERAD pathway and that HRD1 protects cells against the toxic effects of misfolded insulin. In addition, the methods described herein are useful to quantify ER stress level in the islets of Akita mice. There is a high baseline level of ER stress in pancreatic β cells because of the heavy load of client protein, insulin. This means that only a slight increase in ER stress could lead to β-cell death. Thus, the new methods described herein to quantify ER stress level are useful to measure the vulnerability of β cells to ER stress-mediated cell death and can be used for the early diagnosis and prognosis of diabetes. These results indicate not only that HRD1 is upregulated in the diabetes mouse model, but that HRD1 may be central to the protection of β cells from ER stress-mediated death. Thus, small molecules that activate or enhance the HRD1-mediated ERAD pathway are therapeutically beneficial to patients with diabetes.

### Example 11

### IRE1 Activation is Coupled to Insulin Biosynthesis in the Presence of Hyperglycemia

A heavy load of client protein, insulin, causes a high baseline level of ER stress in pancreatic β cells. This means that only a slight increase in ER stress could lead to β-cell death. The major abnormality in patients with type 2 diabetes is peripheral resistance to the action of insulin. This leads to a prolonged increase in insulin biosynthesis in response to elevated glucose level and, because the secretion capacity of the ER is overwhelmed, activates the ER stress signaling pathway. ER stress signaling could lead to the β-cell death associated with hyperglycemia due to insulin resistance. The high levels of ER stress and pancreatic β-cell death in Akita mice may accelerate a process that is played out over years in patients with type 2 diabetes.

IRE1 is a central regulator of ER stress signaling and the ERAD system. It is possible that β-cell apoptosis due to ER stress plays a role in the pathogenesis of type 1 diabetes. Apoptosis of β cells by ER stress may initiate autoimmunity because the engulfment of apoptotic β cells by dendritic cells in the islets may stimulate the β-cell-reactive T cell maturation in draining lymph nodes. Thus, the methods described herein provide new clinical approaches based on the prevention of β-cell death by identifying drugs that block the ER stress-mediated cell-death pathway.

This example describes the results of experiments to evaluate the role of IRE1 activation in insulin biosynthesis.

A. Physiological ER Stress Levels in Mouse Pancreas

To monitor the physiological ER stress level in mouse pancreas, immunoblot analysis and immunohistochemistry of phosphorylated IRE1α were performed using the anti-phospho-specific IRE1α antibody, PIRE1A1, described herein, using lysates from mouse pancreas, prepared as described herein.

Results:

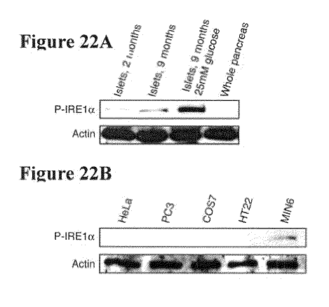

Phosphorylated IRE1α was abundant in the islets, but not in the whole pancreas (FIG. 22A). Immunoblot analyses using lysates from different cell lines showed higher expression of phosphorylated Ire1α in a pancreatic β-cell cell line, MIN6 (FIG. 22B). Immunohistochemistry performed on mouse pancreas using the same antibody detected phosphorylated Ire1α mainly in the islets.

These results indicate that physiological ER stress level is higher in the endocrine cells of the pancreas (i.e., islets) than in exocrine cells, thus suggesting that IRE1 activation and ER stress signaling have an important role in pancreatic β cells.

B. IRE1 Signaling in Insulin Biosynthesis

The majority of cells in islets are β cells, which produce insulin. Thus, a high basal ER stress level in the islets prompted the evaluation of IRE1 signaling involvement in insulin biosynthesis.

Briefly, MIN6 cells, maintained as described herein, were treated with 5 mM or 25 mM of glucose, and insulin biosynthesis and IRE1 phosphorylation levels were measured. Lysates from those cells were subjected to SDS-PAGE. The active form of Ire1α, phospho-Ire1α (P-Ire1α), was detected by immunoblot analysis with anti-phospho specific IRE1α antibody. Cellular expression levels of insulin, protein disulfide isomerase (Pdi), and actin were detected by immunoblot analysis using the same lysates. Insulin secretion level was measured by immunoblot analysis.

INS1 cells were treated with 0 mM, 2.5 mM, 10 mM, 20 mM or 25 mM of glucose and lysates from those cells were subjected to SDS-PAGE. P-IRE1α and insulin were detected by immunoblot analysis.

Results:

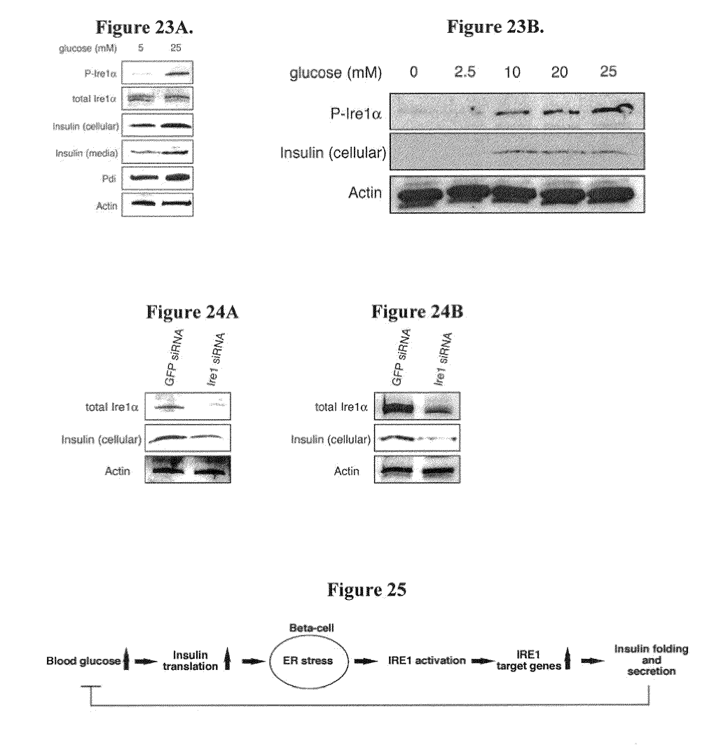

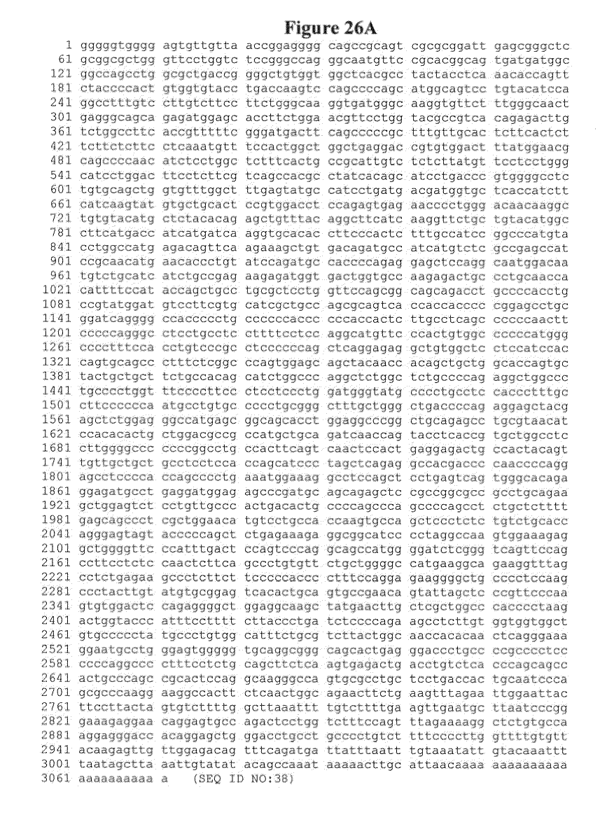

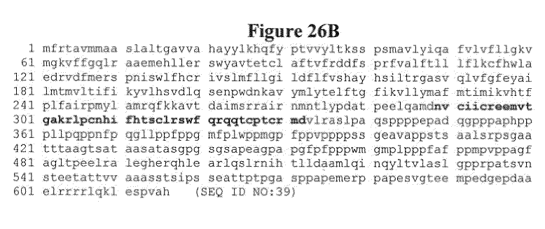

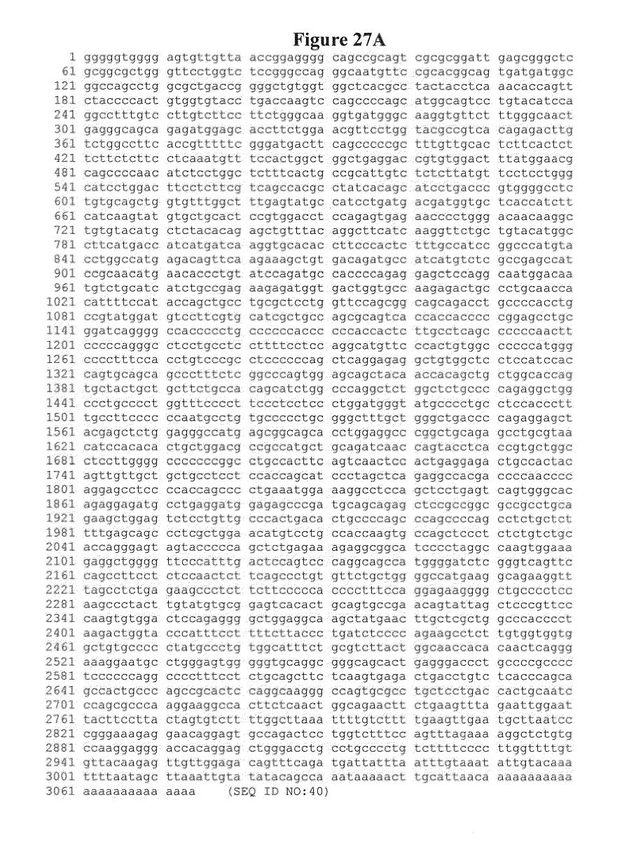

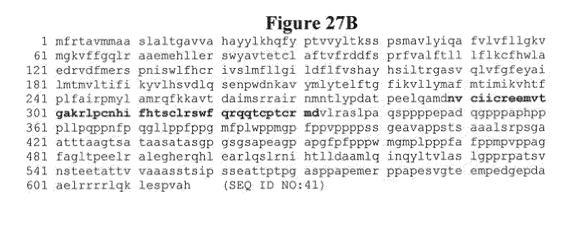

MIN6 cells were treated with 25 mM glucose induced both insulin biosynthesis and IRE1 phosphorylation (FIG. 23A). Treating INS1 cells with 10 mM, 20 mM, and 25 mM of glucose also induced both insulin biosynthesis and IRE1 phosphorylation (FIG. 23B). These results suggest that there is an important relationship between the biosynthesis of insulin and the activation of IRE1 signaling in pancreatic β cells.

C. siRNA Inhibition of IRE1a in MIN6 and INS Cells.

The expression of IRE1α in MIN6 and INS1 cells was knocked out using small interfering RNA (siRNA) specific for IRE1α and decreased insulin biosynthesis.

Duplex 21-mers with dTDT overhangs were used, with the following central target sequences:

| | |
|---|---|
| hIREI α-1: | |
| AAGGCCATGATCTCCGACTTT (for human) | (SEQ ID NO: 35) |
| mIRE1α-1: | |
| AAGGAGCTTTGAGGAAGTTAT (for mouse) | (SEQ ID NO: 36) |
| rIRE1α-1: | |
| AAGGCGATGATCTCAGACTT (for rat) | (SEQ ID NO: 37) |

Results:

Treatment with the siRNA blocked IRE1 protein expression in both cell types (FIGS. 24A and 24B). These results indicate a direct relationship between IRE1 activation and insulin biosynthesis. Thus, IRE1 is a target for controlling insulin synthesis.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A polyclonal antibody PIRE1A1 that binds specifically to an autophosphorylated form of Inositol Requiring 1 (IRE1), and does not substantially bind to an unphosphorylated form of IRE1.

2. A method of determining an endoplasmic reticulum (ER) stress level in a cell or biological sample, the method comprising detecting an Inositol Requiring 1 (IRE1) activity level in the cell or biological sample by detecting the level of autophosphorylated IRE1 using the polyclonal antibody of claim 1, wherein the level of autophosphorylated IRE1 in the cell indicates the stress level in the cell or sample.

3. The method of claim 2, wherein the IRE1 activity level is detected by detecting the ratio of autophosphorylated to unphosphorylated IRE1.

4. The method of claim 2, wherein the ER stress level is determined in a cell.

5. The method of claim 4, wherein the ER stress level is determined in a mammalian cell.

6. The method of any claim 5, wherein the ER stress level is determined in a human cell.

7. The method of claim 4, wherein the cell is a pancreatic beta cell or a peripheral blood cell.

8. The method of claim 2, wherein the ER stress level is determined in a cell extract.

9. A method of identifying a test compound that modulates endoplasmic reticulum (ER) stress, the method comprising:
providing an ER stress model system;
optionally, increasing ER stress in the system;
contacting the system with a test compound; and
evaluating a level of Inositol Requiring 1 (IRE1) activity in the system in the presence and absence of the test compound by detecting a level of autophosphorylated IRE1 in the system using the polyclonal antibody of claim 1, wherein the level of autophosphorylated IRE1 in the cell indicates the level of IRE1 activity in the cell or sample wherein an increase in the level of IRE1 activity indicates that the test compound causes an increase in ER stress, and a decrease in the level of IRE1 activity indicates that the test compound causes a decrease in ER stress.

10. The method of claim 9, wherein the ER stress model system is a cell or animal model of an ER stress disorder.

11. The method of claim 9, wherein ER stress in the system is increased by contacting the system with an agent that increases levels of ER stress.

12. The method of claim 11, wherein the agent that increases ER stress is thapsigargin or tunicamycin.

13. A kit for determining ER stress, the kit comprising the polyclonal antibody of claim 1 and optionally one or both of a control comprising an autophosphorylated form of IRE1 or a control comprising an unphosphorylated form of IRE1, and instructions for use.

14. A composition comprising the polyclonal antibody of claim 1.
